(19) United States
(12) Patent Application Publication
(10) Pub. No.: US 2026/0174429 A1
(43) Pub. Date: Jun. 25, 2026

(54) MULTIPLE NEEDLE SUTURING DEVICE

(71) Applicant: LSI Solutions, Inc., Victor, NY (US)

(72) Inventor: Jude S. Sauer, MD, Pittsford, NY (US)

(21) Appl. No.: 19/187,786

(22) Filed: Apr. 23, 2025

Related U.S. Application Data
(60) Provisional application No. 63/637,749, filed on Apr. 23, 2024.

(51) Int. Cl. A61B 17/04; A61B 17/00

(52) U.S. Cl. CPC A61B 17/0469; A61B 2017/00367; A61B 2017/0472

(57) ABSTRACT

A suturing device includes a housing, an elongated shaft coupled to the housing, and a distal end assembly coupled to a distal end of the shaft. An actuation assembly may include a plurality of actuation levers that each may be rotated from a first position to a second position by grasping a lever portion that extends from the housing. An elongated needle may be coupled to each of the actuation levers such that when the corresponding actuation lever is rotated from the first position to the second position, each needle is displaced from a first needle position, in which the needle tip is proximal to a distal portion of the distal end assembly, to a second needle position, in which the needle tip is disposed within a corresponding portion of the distal portion of the distal end assembly.

### CROSS-REFERENCE TO RELATED APPLICATION

This application claims the benefit of U.S. Provisional Patent Application No. 63/637,749, filed Apr. 23, 2024, which is incorporated by reference herein in its entirety.

### BACKGROUND

The availability of safe and effective therapy for tricuspid valve (TV) disease remains an area of significant unmet clinical need. Tricuspid regurgitation (TR) or the pathologic leakage of blood back into the right atrium during systole, quite common in cardiac patients with left-sided valvular or myocardial disease, is estimated to affect >1.5 million people in the United States, with a yearly incidence of about 200,000 and >300,000 patients in the United States and Europe, respectively. Specific anatomic features from the TV complex might vary according to the causing mechanism (primary vs. secondary) and throughout the progressive stages of ventricular remodeling in patients with functional TR. TR is most often functional, primarily due to annular dilatation and leaflet tethering from right ventricular remodeling caused by left-sided heart disease, atrial fibrillation, or pulmonary hypertension. Primary TR accounts for ^{˜}10 percent of cases of TR and can be due to congenital (Ebstein's anomaly, prolapse) or acquired diseases (rheumatic, endocarditis, carcinoid, endomyocardial fibrosis, intracardiac leads, or bioptome-related iatrogenic trauma). Today, TV disease is often considered a marker for late-stage chronic heart failure. TV is associated with a grim prognosis with most patients receiving lifetime medical therapy until intractable right heart failure and end-organ dysfunction appear.

Secondary TR has been divided into three stages for therapeutic purposes. In the early stage, initial dilation of the right ventricle leads to tricuspid annular dilation without significant leaflet tethering. Annular-based systems should easily repair TR in these first stages. In the absence of long-term durability data for transcatheter TV therapy and on the basis of a surgical predicate, ring may be preferred over suture annuloplasty when possible in order to reduce TR recurrence. In the second stage, progressive right ventricular and tricuspid annular dilation develop, impairing leaflet coaptation. The likelihood for successful Transcatheter Tricuspid Valve Repair (TTVr) using annuloplasty alone is less suitable in cases with progressive tethering and tricuspid annular dilation. Finally, as the right ventricle continues to remodel, further leaflet tethering worsens, resulting in a lack of coaptation and massive or torrential TR. When severe tethering occurs, any repair attempt could be considered futile.

The tricuspid annulus valve is the largest of four heart valves, with very thin, fragile leaflets composing a potentially large regurgitant orifice area. The tricuspid valve is surrounded by the tricuspid valve annulus, a saddle-shaped ellipsoid that becomes planar and circular as it dilates primarily in the anterolateral free wall in patients with left-sided heart disease with sinus rhythm verses expanding mostly along the posterior border with less prominent leaflet tethering in patients with functional TR secondary to chronic atrial fibrillation. The three leaflets, an anterior leaflet, septal leaflet, and posterior leaflet. Four chief anatomic structures surround the TV and are therefore at risk for interventions addressing TV disease: the conduction system (atrioventricular node and the right bundle of His) coursing the membranous septum at 3 to 5 mm from the anteroseptal commissure, the right coronary artery (encircling the right atrioventricular groove-5.5 mm from the septal and posterior portions, 7 mm from the anterior portion), the non-coronary sinus of Valsalva, and the coronary sinus ostium being an important landmark of the posteroseptal commissure. The TV apparatus poses additional challenging issues to overcome: lack of calcium, angulation in relation to the superior vena cava (SVC) and inferior vena cava (IVC), a trabeculated and thin right ventricle hindering a transapical approach, or the presence of pre-existing cardiac implantable electronic devices.

Traditional isolated TV surgery typically requires highly invasive surgical access and cardio-pulmonary by-pass. Since this current approach continues to be associated with one of the highest risks of mortality among all cardiac valve procedures in contemporary practice (operative mortality rates of 8.8 percent to 9.7 percent), it is rarely utilized relative to the large number of untreated TR patients (only 5,005 isolated tricuspid procedures were performed in a large contemporary U.S. nationwide registry over a 10-year period). Durability remains the Achilles heel of most surgical interventions addressing the TV. Many factors, such as right ventricular remodeling and dysfunction, tricuspid annular size progression, and pulmonary hypertension, may contribute to the high rates of TR recurrence observed following surgical TR correction. Surgical experience has shown more sustained durability of ring annuloplasty compared with suture annuloplasty, as well as for TV replacement over repair. However, concerns about increased perioperative mortality for TV replacement compared with repair in contemporary series-somewhat linked to selection bias of patients with larger tricuspid annular dilation and more severe right ventricular dysfunction—have led to a trend over time toward TV repair rather than replacement.

Modern advances in cardiac surgery have made it possible to repair or replace heart valves using minimally invasive surgical techniques. As minimally invasive techniques have improved, surgeons have been able to perform a tricuspid valve repair on patients through smaller and smaller access holes, resulting in less perioperative pain and shorter recovery times. However, due to multiple instruments inserted into the access holes, such as scopes to view the treatment area and a device to suture the leaflets, viewing the treatment area, and maneuvering the distal end of a minimally-invasive device to engage the three leaflets of the tricuspid valve, is difficult. Therefore, it would be desirable to provide a suturing instrument that provide reliable suture placement around a cardiac valve, such as a tricuspid valve, while allowing a scope to be precisely positioned at the treatment area beyond the distal end of the suturing device while minimizing the footprint of the device and scope within the available treatment space. It would be desirable to provide a suturing instrument that would allow the distal end of the device to perform all of the necessary suturing of the delicate leaflets without having to reposition the distal end of the device following each placement of an end of a suture.

### BRIEF DESCRIPTION OF THE DRAWINGS

FIGS. 1A to 1C are side, top, and rear views, respectively, of an embodiment of a suturing device;

FIGS. 1D and 1E are perspective view of the embodiment of the suturing device of FIGS. 1A to 1C;

FIG. 1F is a cross-sectional view of the embodiment of the suturing device of FIGS. 1A to 1C taken along section 1F-1F of FIG. 1B;

FIG. 1G is a cross-sectional view of the embodiment of the suturing device of FIGS. 1A to 1C taken along section 1G-1G of FIG. 1B;

FIG. 1H is a perspective view of the cross-section of FIG. 1G;

FIG. 2A is a cross-sectional view of the embodiment of the suturing device of FIGS. 1A to 1C with a blocking lever in an engaged position;

FIG. 2B is a cross-sectional view of the embodiment of the suturing device of FIGS. 1A to 1C with the blocking lever in a disengaged position;

FIG. 2C is a cross-sectional view of the embodiment of the suturing device of FIGS. 1A to 1C with the blocking lever in the engaged position and engaging a portion of a device;

FIGS. 3A to 3C are side, front, and rear views, respectively of an embodiment of a distal end assembly;

FIG. 3D is a cross-sectional view of the embodiment of the distal end assembly taken along section 3D-3D of FIG. 3A;

FIG. 3E is a cross-sectional view of the embodiment of the distal end assembly taken along section 3E-3E of FIG. 3A;

FIG. 3F is a cross-sectional view of the embodiment of the distal end assembly taken along section 3F-3F of FIG. 3A;

FIGS. 4A to 4C are perspective views of the distal end FIGS. 3A to 3C;

FIG. 4D is a cross-sectional view of a distal end of the embodiment of the suturing device of FIGS. 1A to 1C taken along section 1F-1F of FIG. 1B;

FIGS. 5A to 5E are various views of an embodiment of an actuation assembly;

FIGS. 6A to 6E are various views of an embodiment of the housing of the embodiment of the actuation assembly;

FIGS. 7A to 7E are various views of an embodiment of a blocking lever;

FIG. 8 is a detailed rear view of the embodiment of the suturing device of FIGS. 1A to 1C;

FIGS. 9A to 9D are various views of an embodiment of a cam member of a position adjustment assembly of the suturing device of FIGS. 1A to 1C;

FIGS. 9E and 9F are cross-sectional views of the cam member taken along section 9E-9E of FIG. 9B;

FIGS. 9G and 9H are cross-sectional views of the cam member taken along section 9G-9G of FIG. 9A;

FIGS. 10A and 10B are various views of an embodiment of a collar of a position adjustment assembly of the suturing device of FIGS. 1A to 1C;

FIG. 10C is a cross-sectional view of the collar taken along section 10C-10C of FIG. 10A;

FIG. 11 is a cross-sectional view of a portion of the position adjustment assembly, with the shaft and other elements omitted for clarity, taken along section 11-11 of FIG. 1B;

FIGS. 12A to 12C are various views of an embodiment of a cam follower member of the position adjustment assembly of the suturing device of FIGS. 1A to 1C;

FIGS. 13A and 13B are cross-sectional views of a portion of the position adjustment assembly, with the shaft and other elements omitted for clarity, taken along section 11-11 of FIG. 1B;

FIGS. 14A to 14C are various views of an embodiment of a hinge portion of the position adjustment assembly of the suturing device of FIGS. 1A to 1C;

FIG. 14D is a cross-sectional view of the hinge portion taken along section 14D-14D of FIG. 14A; and

FIGS. 15A to 15J are various views of a method of using the suturing device to repair a tricuspid valve of a heart.

### DETAILED DESCRIPTION OF THE INVENTION

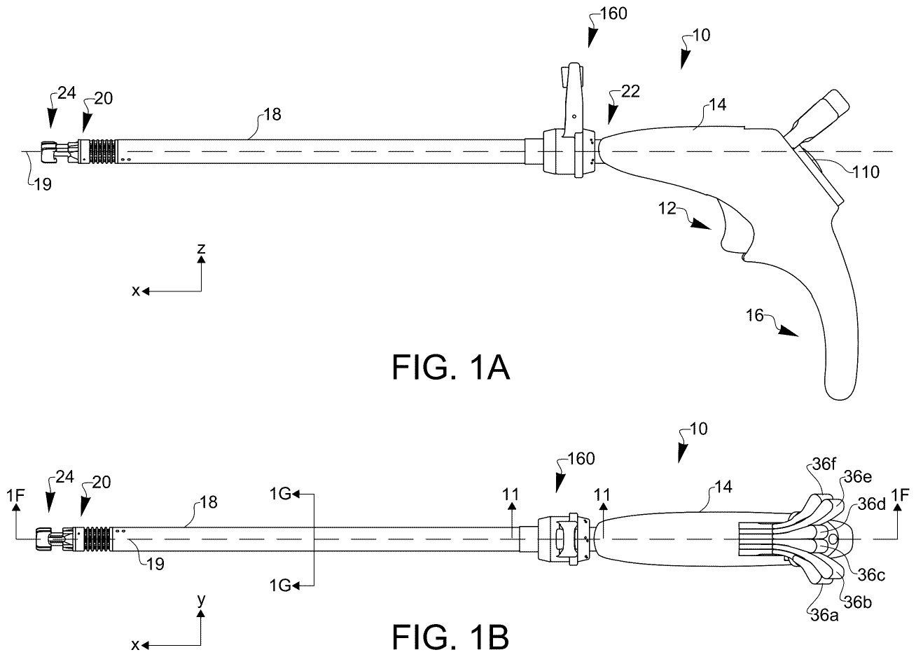

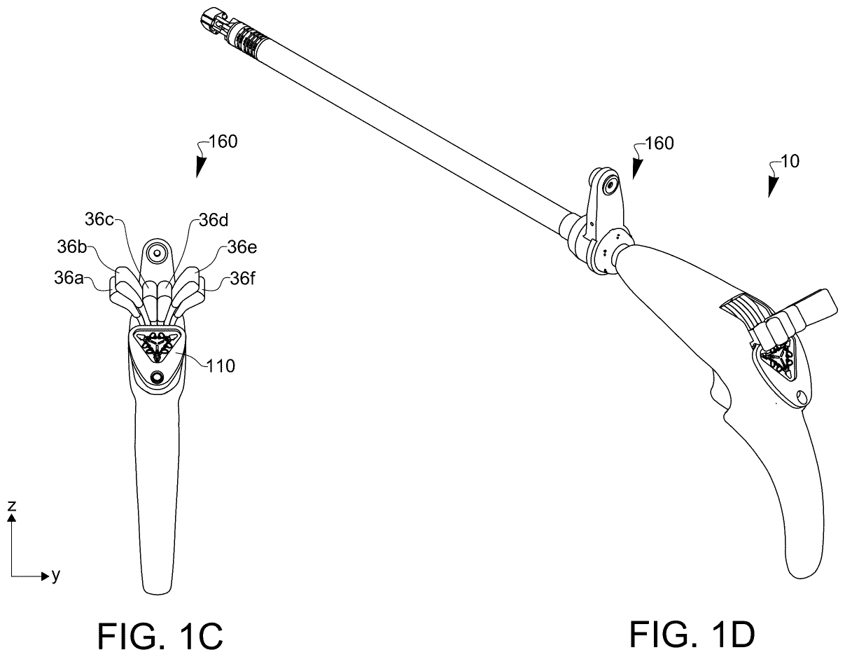

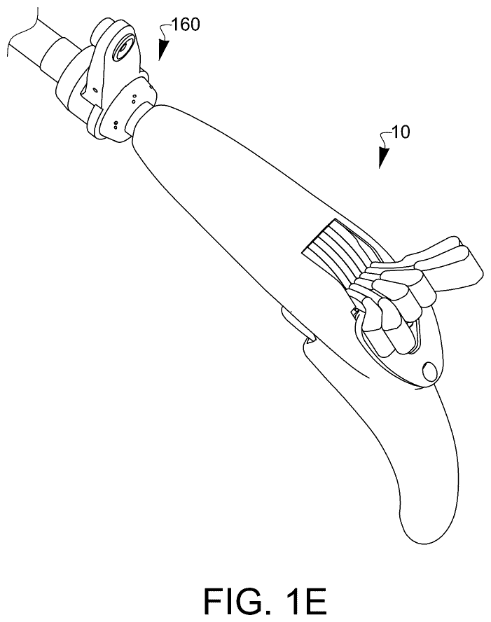

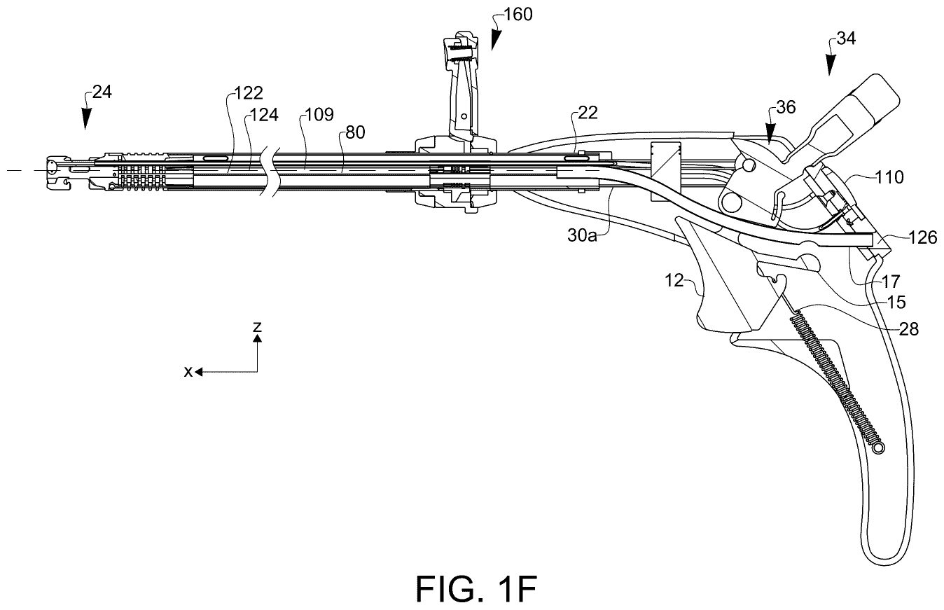

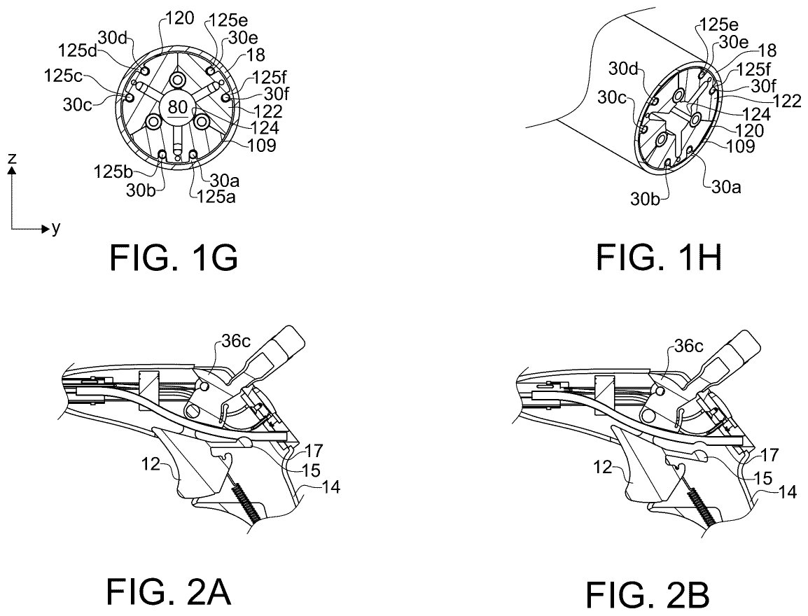

FIGS. 1A and 1B illustrate an embodiment of a suturing device 10 which may be used to, inter alia, allow for the precise placement of a scope. such as an endoscope or an ICE (intracardiac echo) probe, in a manner that maximizes the available space around a treatment area and that also allows for the placement of multiple sutures in the delicate leaflets of the tricuspid valve during a repair procedure without the need to significantly reposition the suturing device 10 after each suture is placed.

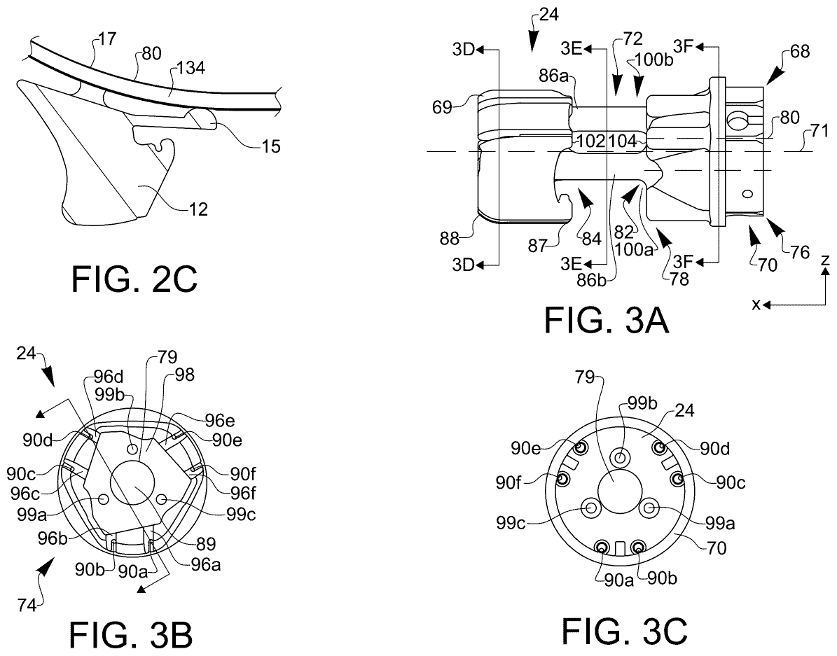

Turning to the side view of FIG. 1A, the suturing device 10 includes the housing portion 14 having a grip portion 16 that is adapted to be grasped by a user to position a distal end 20 of the shaft 18 of the suturing device 10 during a procedure. As illustrated in FIGS. 2A and 2B, in which a portion of the housing portion 14 is omitted for clarity, the grip portion 16 can also be adapted to be grasped by the user to engage and displace a blocking lever 12 from a disengaged position (illustrated in FIG. 2B) to an engaged position (illustrated in FIG. 2A). In the disengaged position, an engagement portion 15 of an engagement arm 21 of the blocking lever 12 is disposed remote from a portion of an access tube 17 (illustrated in FIG. 2B), such as an aperture in the access tube 17, and the engagement portion 15 of the blocking lever 12 and the access tube 17 will both be discussed in more detail in a following section. In some embodiments, the engagement portion 15 may be disposed at or adjacent to an end of the engagement arm 21 of the blocking lever 12. In the engaged position, a portion of the engagement portion 15 of the blocking lever 12 may be inserted into the portion of the access tube 17 (illustrated in FIG. 2A), such as within the aperture in the access tube 17, to frictionally engage a portion of a device, such as an endoscope or an ICE (intracardiac echo) probe 134 (see FIG. 2C), that is disposed within the access tube 17, thereby preventing further movement of the device within the access tube 17.

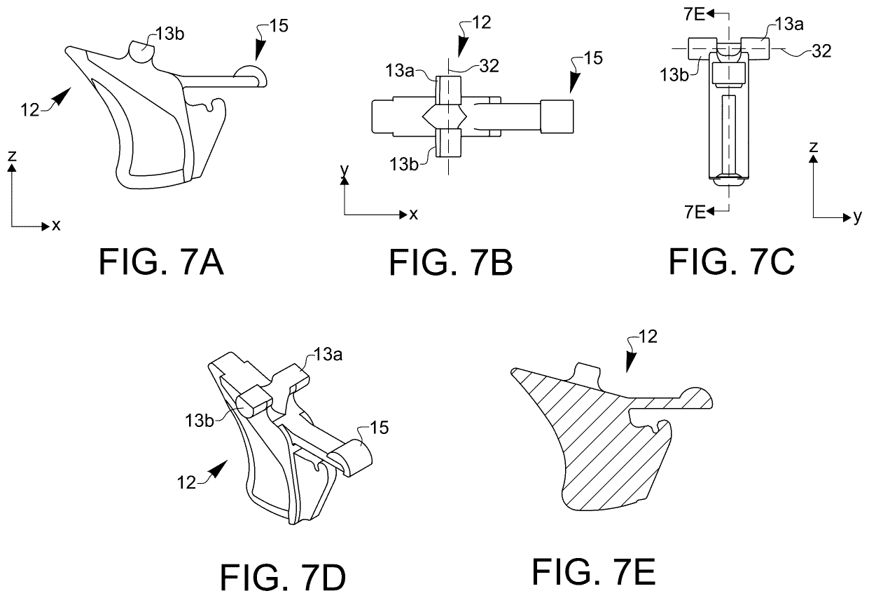

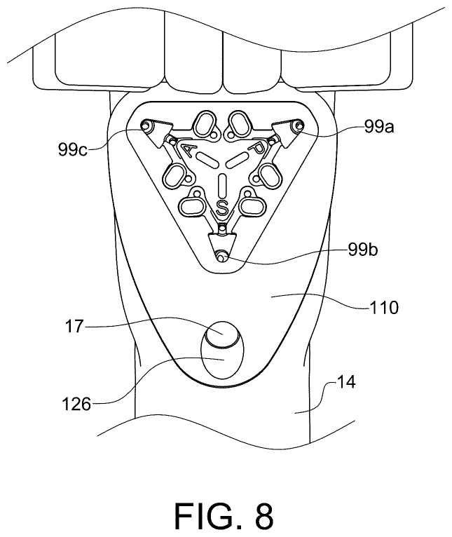

With reference to FIG. 7D, a first portion 13a of the blocking lever 12 may be rotatably coupled to the housing portion 14 at a first pivot portion of the housing portion 14 and a second portion 13b of the blocking lever 12 may be rotatably coupled to the housing portion 14 at a second pivot portion of the housing portion 14. The first portion 13a and the second portion 13b of the blocking lever 12 are configured such that the blocking lever 12 pivots between the first lever position to the second lever position about a pivot axis 32, which is illustrated in FIGS. 7B and 7C. In some embodiments, the first portion 13a of the blocking lever 12 may include a first of a pair of aligned bosses that may be received into corresponding cylindrical internal walls (not shown) that are disposed at the first pivot portion of the housing portion 14. The second portion 13b of the blocking lever 12 may further include a second of the pair of aligned bosses that may be received into corresponding cylindrical internal walls (not shown) that are disposed at the second pivot portion of the housing portion 14. So disposed, a rotational axis of each of the first portion 13a and the second portion 13b of the blocking lever 12 are coaxially aligned with the pivot axis 32, and the pivot axis 32 may be normal to a shaft axis 19 (see FIG. 1B) that extends along the shaft 18 that is coupled to the housing portion 14. A first end of a spring 28 may be coupled to a portion of the blocking lever 12 and a second end of the spring 28 may be coupled to a portion of the interior portion of the housing portion 14 such that the blocking lever 12 is biased into the first lever position by the spring 28.

Referring to FIG. 1A, the suturing device 10 includes the shaft 18 that extends from a proximal end 22 to a distal end 20 along the shaft axis 19, and a portion of the shaft 18 (e.g., a portion of the proximal end 22 of the shaft 18) may extend into the interior of the housing portion 14. The shaft axis 19 may be linear from the proximal end 22 to the distal end 20, and the shaft axis 19 may extend or generally extend in a direction along or parallel to the X-axis of the reference coordinate system of FIG. 1A. In some embodiments, one or more portions of the shaft 18 may be non-linear. In other embodiment, one or more portions of the shaft 18 may extend along the shaft axis 19, and one of more portions of the shaft 18 may be linear but may extend at an angle to the shaft axis 19. The shaft 18 may be rigid, but in other embodiments, the shaft 18 may be flexible or may have one or more portions that are flexible.

The shaft 18, or one or more portions of the shaft 18, may have the general shape of an elongated hollow tube having an interior surface 120 (illustrated in FIG. 1G) that defines an interior portion 109 that extends from the proximal end 22 to the distal end 20 of the shaft 18. The shaft 18 and the interior surface 120 may have any suitable cross-sectional shape or combination of shapes normal to the shaft axis 19. For example, the shaft 18 may have the general shape of an elongated cylinder, and the interior surface 120 may have a circular cross-sectional shape when viewed normal to the shaft axis 19.

The suturing device 10 may also include an actuation assembly 34 that includes two or more actuation levers 36, as illustrated in FIG. 1F. While the embodiment of FIGS. 1A to 1C includes a first actuation lever 36a, a second actuation lever 36b, a third actuation lever 36c, a fourth actuation lever 36d, a fifth actuation lever 36e, and a sixth actuation lever 36f, other embodiments may include only a first actuation lever 36a and a second actuation lever 36b, or a first actuation lever 36a, a second actuation lever 36b, a third actuation lever 36c, and a fourth actuation lever 36d. Other embodiments may have a seventh actuation lever and an eighth actuation lever, etc.

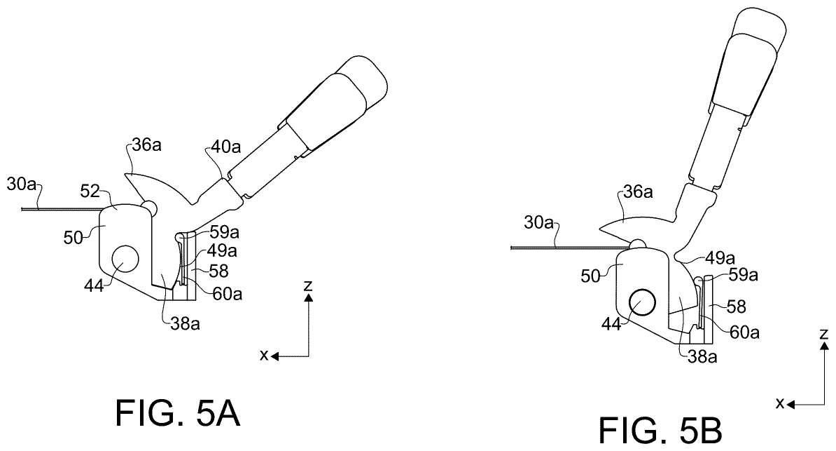

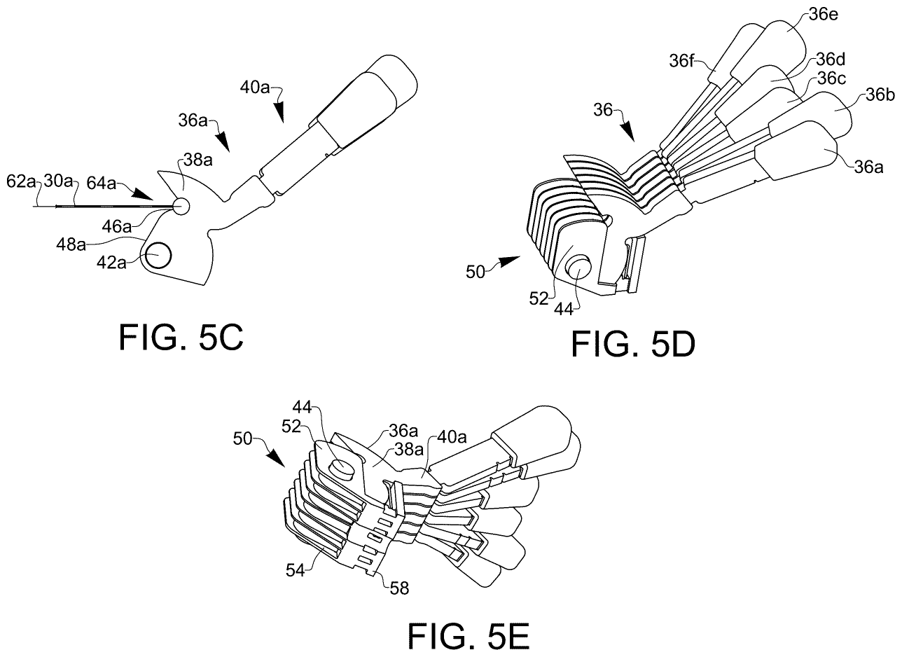

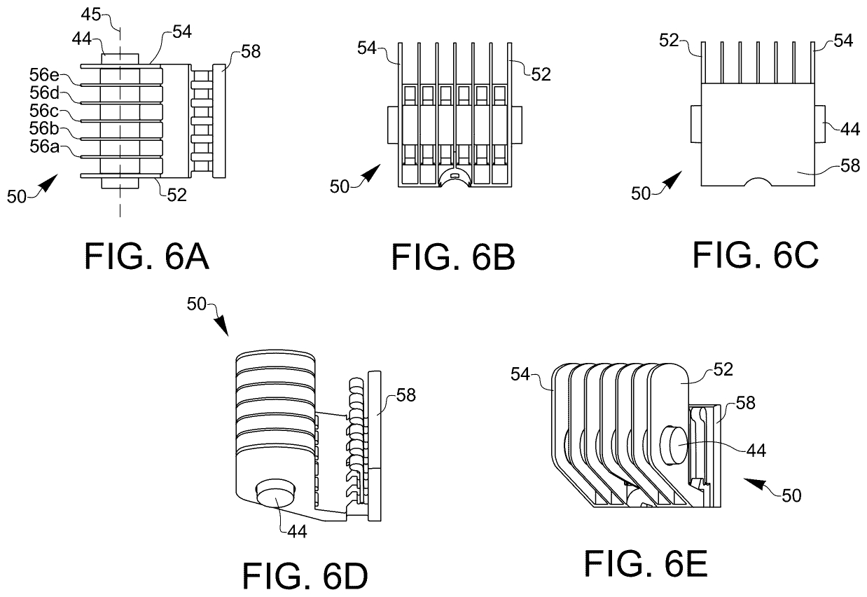

In particular, the actuation assembly 34 may include the first actuation lever 36a, which is illustrated in FIG. 5C. The first actuation lever 36a may include a base portion 38a that may be planar or substantially planar, and a handle portion 40a may extend from a first end portion of the base portion 38a. In some embodiments, the handle portion 40a may be planar of substantially planar and may be integrally formed with the base portion 38a such that the base portion 38a and the handle portion 40a may be formed from one or more stamping and/or cutting processes from a single piece of stock material. A pin aperture 42a may be formed at a second end portion of the base portion 38a, and the pin aperture 42a may receive a pin 44 (see FIGS. 5A, 5B, 5D, and 5E) that extends along a longitudinal axis 45 (see FIG. 6A), and the longitudinal axis 45 of the pin 44 may be normal to the shaft axis 19. The first actuation lever 36a may pivot about a first portion of the pin 44, and the axis of rotation may be the longitudinal axis 45 of the pin 44 such that the base portion 38a rotates in a plane along or parallel to the X-Z plane of the reference coordinate system of FIGS. 1A to 1C. The base portion 38a may further include a connection portion 46a that may be disposed along a portion of the base portion 38a disposed along a front edge 48a of the base portion 38a, and the connection portion 46a may be disposed offset from the pin aperture 42a.

As illustrated in FIG. 5D, the actuation assembly 34 may include the second actuation lever 36b that may be identical or substantially identical to the first actuation lever 36a. The second actuation lever 36b may be offset from the first actuation lever 36a along or parallel to the longitudinal axis 45 of the pin 44 and/or the Y-axis of the reference coordinate system of FIGS. 1A to 1C. The second actuation lever 36b may include a base portion 38b that may be planar or substantially planar, and a handle portion 40b may extend from a first end portion of the base portion 38b. A pin aperture 42b may be formed at a second end portion of the base portion 38b, and the second actuation lever 36b may pivot about a second portion of the pin 44 such that the base portion 38b rotates in a plane along or parallel to the X-Z plane of the reference coordinate system of FIGS. 1A to 1C. The base portion 38b may further include a connection portion 46b that may be disposed along a portion of the base portion 38b disposed along a front edge 48b of the base portion 38b, and the connection portion 46b may be disposed offset from the pin aperture 42b.

As illustrated in FIG. 5D, the actuation assembly 34 may include the third actuation lever 36c that may be identical or substantially identical to the first actuation lever 36a. The third actuation lever 36c may be offset from the second actuation lever 36b along or parallel to the longitudinal axis 45 of the pin 44 and/or the Y-axis of the reference coordinate system of FIGS. 1A to 1C, and the second actuation lever 36b may be disposed between the third actuation lever 36d and the first actuation lever 36a. The third actuation lever 36c may include a base portion 38c that may be planar or substantially planar, and a handle portion 40c may extend from a first end portion of the base portion 38c. A pin aperture 42c may be formed at a second end portion of the base portion 38c, and the third actuation lever 36c may pivot about a third portion of the pin 44 such that the base portion 38c rotates in a plane along or parallel to the X-Z plane of the reference coordinate system of FIGS. 1A to 1C. The base portion 38c may further include a connection portion 46c that may be disposed along a portion of the base portion 38c disposed along a front edge 48c of the base portion 38c, and the connection portion 46c may be disposed offset from the pin aperture 42c.

As illustrated in FIG. 5D, the actuation assembly 34 may include the fourth actuation lever 36d that may be identical or substantially identical to the first actuation lever 36a. The fourth actuation lever 36d may be offset from the third actuation lever 36c along or parallel to the longitudinal axis 45 of the pin 44 and/or the Y-axis of the reference coordinate system of FIGS. 1A to 1C, and the third actuation lever 36c may be disposed between the fourth actuation lever 36d and the second actuation lever 36b. The fourth actuation lever 36d may include a base portion 38d that may be planar or substantially planar, and a handle portion 40d may extend from a first end portion of the base portion 38d. A pin aperture 42d may be formed at a second end portion of the base portion 38d, and the fourth actuation lever 36d may pivot about a fourth portion of the pin 44 such that the base portion 38d rotates in a plane along or parallel to the X-Z plane of the reference coordinate system of FIGS. 1A to 1C. The base portion 38d may further include a connection portion 46d that may be disposed along a portion of the base portion 38d disposed along a front edge 48d of the base portion 38d, and the connection portion 46d may be disposed offset from the pin aperture 42d.

As illustrated in FIG. 5D, the actuation assembly 34 may include the fifth actuation lever 36e that may be identical or substantially identical to the first actuation lever 36a. The fifth actuation lever 36e may be offset from the fourth actuation lever 36d along or parallel to the longitudinal axis 45 of the pin 44 and/or the Y-axis of the reference coordinate system of FIGS. 1A to 1C, and the fourth actuation lever 36d may be disposed between the fifth actuation lever 36e and the third actuation lever 36c. The fifth actuation lever 36e may include a base portion 38e that may be planar or substantially planar, and a handle portion 40e may extend from a first end portion of the base portion 38e. A pin aperture 42e may be formed at a second end portion of the base portion 38e, and the fifth actuation lever 36e may pivot about a fifth portion of the pin 44 such that the base portion 38e rotates in a plane along or parallel to the X-Z plane of the reference coordinate system of FIGS. 1A to 1C. The base portion 38e may further include a connection portion 46e that may be disposed along a portion of the base portion 38e disposed along a front edge 48e of the base portion 38e, and the connection portion 46e may be disposed offset from the pin aperture 42e.

As illustrated in FIG. 5D, the actuation assembly 34 may include the sixth actuation lever 36f that may be identical or substantially identical to the first actuation lever 36a. The sixth actuation lever 36f may be offset from the fifth actuation lever 36e along or parallel to the longitudinal axis 45 of the pin 44 and/or the Y-axis of the reference coordinate system of FIGS. 1A to 1C, and the fifth actuation lever 36e may be disposed between the sixth actuation lever 36f and the fourth actuation lever 36d. The sixth actuation lever 36f may include a base portion 38f that may be planar or substantially planar, and a handle portion 40f may extend from a first end portion of the base portion 38f. A pin aperture 42f may be formed at a second end portion of the base portion 38f, and the sixth actuation lever 36f may pivot about a sixth portion of the pin 44 such that the base portion 38f rotates in a plane along or parallel to the X-Z plane of the reference coordinate system of FIGS. 1A to 1C. The base portion 38f may further include a connection portion 46f that may be disposed along a portion of the base portion 38f disposed along a front edge 48f of the base portion 38f, and the connection portion 46f may be disposed offset from the pin aperture 42f.

The actuation assembly 34 may also include a housing 50, which is illustrated in FIGS. 6A to 6C, with the actuation levers 36 omitted for clarity. As illustrated in FIG. 6A, a first end of the pin 44 may be coupled with a first outer lateral wall 52 of the housing 50 and a second end of the pin 44 may be coupled with a second outer lateral wall 54 of the housing 50. The housing 50 may also include a plurality of separation walls 56 disposed between the first outer lateral wall 52 and the second outer lateral wall 54. Each of the separation walls 56 is configured to be disposed between adjacent actuation levers 36, and each of the separation walls 56 may be planar and configured to be parallel to the base portions 38 of the adjacent actuation levers 36 such that each of the separation walls 56 may extend in a plane along or parallel to the X-Z plane of the reference coordinate system of FIGS. 1A to 1C. In the embodiment of FIG. 6A, a first separation wall 56a may be disposed between the first actuation lever 36a and the second actuation lever 36b, a second separation wall 56b may be disposed between the second actuation lever 36b and the third actuation lever 36c, a third separation wall 56c may be disposed between the third actuation lever 36c and the fourth actuation lever 36d, a fourth separation wall 56d may be disposed between the fourth actuation lever 36d and the fifth actuation lever 36e, a fifth separation wall 56e may be disposed between the fifth actuation lever 36e and the sixth actuation lever 36f.

The housing 50 may also include a rear wall 58 that may extend between the first outer lateral wall 52 and the second outer lateral wall 54, and a portion of the rear wall 58 may be configured to contact a portion of the base portion 38 of a corresponding actuation lever 36 to limit rotation. The portion may be disposed along a rear edge 49 of the base portion 38. For example, FIG. 5A illustrates the first actuation lever 36a having a rear edge 49a, and when the first actuation lever 36a is pivoted from a second lever position (illustrated in FIG. 5B) to a first lever position (illustrated in FIG. 5A), a first portion of the rear edge 49a of the base portion 38a may contact the portion of the rear wall 58 to prevent further clockwise rotation of the first actuation lever 36a about the pin 44. In this first lever position in FIG. 5A, an end portion 59a of a first elongated member 60a may engage a second portion of the rear edge 49a of the base portion 38a to prevent further clockwise rotation of the first actuation lever 36a about the pin 44. In some embodiments, the second portion of the rear edge 49a of the base portion 38a may be contoured to receive a correspondingly contoured shape of the end portion 59a of the first elongated member 60a in the first lever position. The housing 50 may additionally include an elongated member to correspond to each actuation lever 26 such as a second elongated member 60b to engage the second actuation lever 36b in the first lever position, a third elongated member 60c to engage the third actuation lever 36c in the first lever position, a fourth elongated member 60d to engage the fourth actuation lever 36d in the first lever position, a fifth elongated member 60e to engage the fifth actuation lever 36e in the first lever position, and a sixth elongated member 60f to engage the sixth actuation lever 36f in the first lever position.

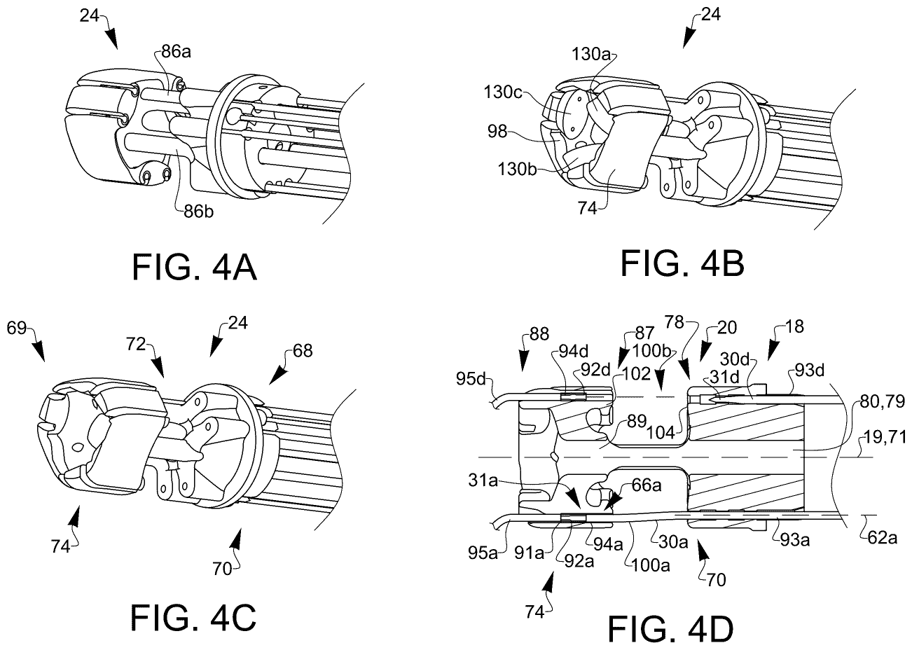

With reference to FIG. 6A, the suturing device 10 includes a needle 30 that is coupled to the connection portion 46 of each actuation lever 36. Each needle 30 may be identical to a first needle 30a, which is illustrated in FIG. 5C, and the first needle 30a may extend along a first needle axis 62a from a proximal end 64a to a distal end 66a (illustrated in FIG. 4D), and the first needle axis 62a may be linear. At least a portion of the first needle 30a extends through the interior portion 109 of the shaft 18 and the proximal end 64a may extend into the interior of the housing portion 14. As illustrated in FIG. 4D, a first needle tip 31a may be disposed at the distal end 66a of the first needle 30a, and the first needle tip 31a may converge to a sharp point such that the distal end 66a is configured and shaped to pierce tissue that may be disposed within a portion the distal end assembly 24 when the first needle 31a extends from a first needle position (i.e., a retracted position illustrated in FIGS. 1F and 4A to 4C) to a second needle position (i.e., an extended position illustrated in FIG. 4D), as will be described in more detail in a following section. As illustrated in FIG. 5C, the proximal end 64a of the first needle 30a may be (or may be configured to be) coupled to the first connection portion 46a of the first actuation lever 36a such that when the first actuation lever 36a is in the first lever position of FIG. 5A, the first needle 30a is in the first needle position. As the first actuation lever 36a is rotated from the first lever position towards the second lever position of FIG. 5B, the first needle 30a is distally displaced along the first needle axis 62a until the first actuation lever 36a is displaced to the second lever position, in which the first needle 30a is in the second needle position. Conversely, when the first actuation lever 36a is pivoted from the second lever position towards the first lever position, the first needle 30a displaces proximally along the first needle axis 62a from the second needle position towards the first needle position, and as the first actuation lever 36a reaches the first lever position, the first needle 30a is disposed in the first needle position. An identical second needle 30b may be coupled to the second actuation lever 36b, a third needle 30c may be coupled to the third actuation lever 36c, a fourth needle 30d may be coupled to the fourth actuation lever 36d, a fifth needle 30e may be coupled to the fifth actuation lever 36e, and a sixth needle 30f may be coupled to the sixth actuation lever 36f.

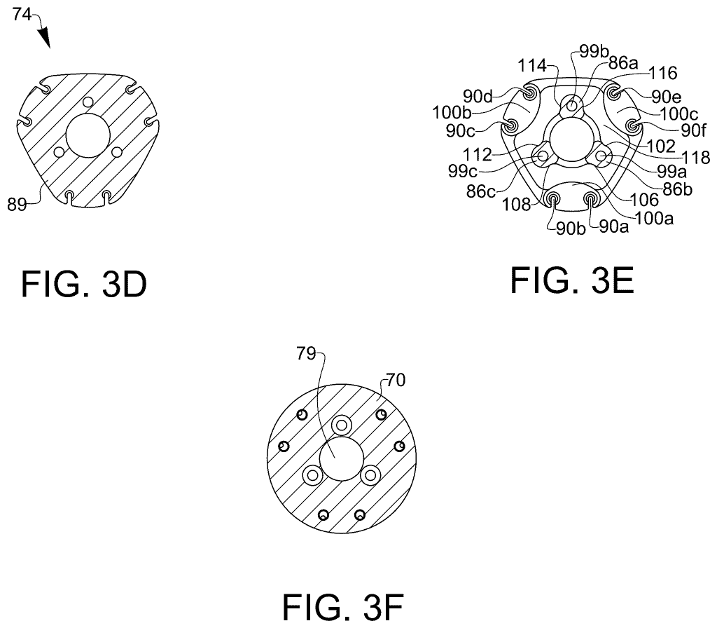

With reference to FIG. 1A, the suturing device 10 may include the distal end assembly 24 coupled or fixedly secured to the distal end 20 of the shaft 18 or a point adjacent to the distal end 20 of the shaft 18. With refence to FIGS. 3A to 3F, the distal end assembly 24 may extend along or generally along a longitudinal axis 71 that may be parallel to the shaft axis 19, and the longitudinal axis 71 may be parallel to the X-axis of the reference coordinate system of FIGS. 1A and 1B. The distal end assembly 24 may extend along the longitudinal axis from a proximal end 68 of the distal end assembly 24 to a distal end 69 of the distal end assembly 24, and all or a portion of the proximal end 68 may be inserted within interior portion 109 of the shaft 18 and may be disposed at or adjacent to the distal end 20 of the shaft 18. The distal end assembly 24 may include a base portion 70, a support portion 72, and a receiver portion 74.

As illustrated in FIG. 3A, the base portion 70 of the distal end assembly 24 may extend from a proximal end 76 (illustrated in FIG. 3C) to a distal end 78 along the longitudinal axis 71, and the proximal end 76 may be disposed at the proximal end 68 of the distal end assembly 24. The base portion 70 may be cylindrical, and may have a cylindrical aperture 79 that may be part of, or may comprise a portion of, an instrument conduit 80 (see FIG. 1F) that may be configured to receive an instrument, such as an endoscope or an ICE probe. A longitudinal axis of the cylindrical aperture 79 may extend along the longitudinal axis 71 of the distal end assembly 24.

Still referring to FIG. 3A, the support portion 72 of the distal end assembly 24 may be disposed between the base portion 70 and the receiver portion 74. The support portion 72 may extend from a proximal end 82 to a distal end 84 along the longitudinal axis 71 such that the proximal end 82 of the support portion 72 is disposed at the distal end 78 of the base portion 70. The support portion 72 may include one or more columns 86, and in some embodiments, three columns 86a, 86b, 86c may be symmetrically arrayed around the cylindrical aperture 79 of the base portion so as to not obstruct the cylindrical aperture 79, and each column 86a, 86b, 86c may be separated from the adjacent column 86a, 86b, 86c by an angle of 120°. Each of the columns 86a, 86b, 86c may extend parallel to the longitudinal axis 71 from the proximal end 82 of the support portion 72 to the distal end 84 of the support portion 72, and a portion of an outer surface of each of the columns 86a, 86b, 86c may cooperate to comprise a portion of the instrument conduit 80. The columns 86a, 86b, 86c

Referring again to FIG. 3A, the distal end assembly 24 may include the receiver portion 74 disposed and/or at or adjacent to the distal end 69 of the distal end assembly 24. In particular, the receiver portion 74 may extend from a proximal end 87 to a distal end 88 along the longitudinal axis 71, and the proximal end 87 of the receiver portion 74 may be disposed at or adjacent to the distal end 84 of the support portion 72. The receiver portion 74 may include a cylindrical aperture 89 that may comprise a portion of the instrument conduit 80, and a longitudinal axis of the cylindrical aperture 89 may extend along the longitudinal axis 71 of the distal end assembly 24.

As illustrated in FIG. 3B, the distal end assembly 24 may include a needle aperture 90 that corresponds to each of the needles 30, and each needle aperture 90 may be configured to receive a portion of one of the needles 30 of the suturing device 10. Each needle aperture 90 may extend through the base portion 70 and all or a portion of the receiver portion 74 along an axis that is aligned with the corresponding needle axis 62 and is parallel to the longitudinal axis 71. For example, as illustrated in the cross-sectional view of the distal end assembly of FIG. 4D, the distal end assembly 24 may include a first needle aperture 90a that is aligned with and configured to receive a portion of the first needle 30a, and the first needle aperture 90a may be aligned with the first needle axis 62a. A proximal portion 93a of the first needle aperture 90a may extend through a first portion of the base portion 70 and a distal portion 94a of the first needle aperture 90a may extend through a first portion of the receiver portion 74. So configured, when the first needle 30a is in the first needle position of FIG. 1F, the first needle tip 31a at the distal end 66a of the first needle 30a may be disposed within the proximal portion 93a of the first needle aperture 90a and may be disposed proximal to the distal end 78 of the base portion 70. Further, when the first needle 30a is displaced from the first needle position to the second needle position of FIG. 4D, the first needle 30a, and the first needle tip 31a, extends distally along the first needle axis 62a until the first needle tip 31a is disposed within the distal portion 94a of the first needle aperture 90a disposed in the receiver portion 74. When the first needle 30a is in the second needle position, the first needle tip 31a is disposed distal to the proximal end 87 of the receiver portion 74.

When the first needle 30a extends from the first needle position to the second needle position, the first needle tip 31a extends through a first tissue bite area 100a, illustrated in FIGS. 3A and 3E. The first tissue bite area 100a may be partially defined by a first portion of a proximal transverse surface 102 of the receiver portion 74 disposed at the proximal end 87 of the receiver portion 74. The first tissue bite area 100a may be further defined by a first portion of a distal transverse surface 104 of the base portion 70 disposed at the distal end 78 of the base portion 70. The first tissue bite area 100a may be further defined by a first lateral surface 106 of the second column 86b and a first lateral surface 108 of the third column 86c. So configured, when a first portion of tissue or synthetic material may be disposed within the first tissue bite area 100a, the first needle tip 31a of the first needle 30a may extend through the first portion of tissue or synthetic material when the first needle 30a extends from the first needle position to the second needle position.

Also in the second needle position, the first needle tip 31a may be disposed within a first ferrule recess 91a that is formed in all or a portion of the distal portion 94a of the first needle aperture 90a disposed in the receiver portion 74, and when the first needle tip 31a is disposed within the first ferrule recess 91a, the first needle tip 31a operatively engages (or is configured to engage) a first ferrule 92a that is disposed in the first ferrule recess 91a. The first ferrule 92a may be any ferrule that is configured to be secured to a first portion of suture 95a extending from a distal end of the first ferrule 92a and that may be configured to be selectively coupled to and uncoupled from the first needle tip 31a. For example, the first ferrule 92a may be configured to be selectively coupled to and uncoupled from the first needle tip 31a via an interface (i.e., a channel) that inwardly extends from a proximal end of the first ferrule 92a, and the first ferrule 92a may be similar to the ferrule disclosed in U.S. Pat. No. 10,390,818, which issued on Aug. 27, 2019 and which is herein incorporated by reference in its entirety.

The first ferrule recess 91a may be sized and configured to retain the first ferrule 92a when the first needle tip 31a of the first needle 30a is in the first needle position, but is configured to allow the first ferrule 92a to operatively couple to the first needle tip 31a of the first needle 30a when the first needle 30a displaces from the second needle position to the first needle position, thereby pulling the first ferrule 92a (and the first portion of suture 95a coupled to the first ferrule 92a) through a first aperture formed in the first portion of tissue or synthetic material disposed within the first tissue bite area 100a. One or more locking or release mechanisms may be disposed at or adjacent to (and/or remote from) the first ferrule recess 91a to selectively retain and release the first ferrule 92a.

With reference to FIG. 3B, a first suture channel 96a may be disposed in a first portion of a distal transverse surface 98 of the receiver portion 74, and the first suture channel 96a may extend from a distal end of the distal portion 94a of the first needle aperture 90a to a distal end of a first suture aperture 99a, and a portion of the first portion of suture 95a may be disposed within the first suture channel 96a such that a second portion of the first portion of suture 95a may extend through the first suture aperture 99a. A third portion of the first portion of suture 95a may extend through a proximal end of the first suture aperture 99a that may be disposed at a proximal portion of the housing portion 14. A plate 110 may be disposed at the proximal portion of the housing portion 14, and the plate may be disposed adjacent to the actuation levers 36 such that a user actuating the actuation levers 36 may have a view of the plate 110. The proximal end of the first suture aperture 99a may be disposed at a corresponding aperture in the plate 110, and a fourth portion of the first portion of suture 95a may be disposed along or across a first portion of the plate 110 so that the user may be able to determine the tension of the first portion of suture 95a by a visual inspection of the fourth portion of the first portion of suture 95a. The fourth portion of the first portion of suture 95a may also be disposed offset from first portion of the plate 110 for the user to determine the tension of the first portion of suture 95a by a visual inspection.

As illustrated in FIG. 3B, the distal end assembly 24 may include a second needle aperture 90b, and the second needle aperture 90b may be identical to, and radially offset from, the first needle aperture 90a. That is, the second needle aperture 90b may be aligned with and configured to receive a portion of the second needle 30b, and the second needle aperture 90b may be aligned with the second needle axis 62b. A proximal portion 93b of the second needle aperture 90b may extend through a second portion of the base portion 70 and a distal portion 94b of the second needle aperture 90b may extend through a second portion of the receiver portion 74. So configured, when the second needle 30b is in the first needle position of FIG. 1F, the second needle tip 31b at the distal end 66b of the second needle 30b may be disposed within the proximal portion 93b of the second needle aperture 90b and may be disposed proximal to the distal end 78 of the base portion 70. Further, when the second needle 30b is displaced from the first needle position to the second needle position of FIG. 4D, the second needle 30b, and the second needle tip 31b, extends distally along the second needle axis 62b until the second needle tip 31b is disposed within the distal portion 94b of the second needle aperture 90b disposed in the receiver portion 74. When the second needle 30b is in the second needle position, the second needle tip 31b is disposed distal to the proximal end 87 of the receiver portion 74.

In the second needle position, the second needle tip 31b may be disposed within a second ferrule recess 91b that is formed in all or a portion of the distal portion 94b of the second needle aperture 90b disposed in the receiver portion 74, and when the second needle tip 31b is disposed within the second ferrule recess 91b, the second needle tip 31b operatively engages (or is configured to engage) a second ferrule 92b that is disposed in the second ferrule recess 91b. The second ferrule recess 91b and the second ferrule 92b may be identical to the first ferrule recess 91a and the first ferrule 92a, respectively. That is, the second ferrule 92b may be any ferrule that is configured to be secured to a second portion of suture 95b extending from a distal end of the second ferrule 92b and that may be configured to be selectively coupled to and uncoupled from the second needle tip 31b.

The second ferrule recess 91b may be sized and configured to retain the second ferrule 92b when the second needle tip 31b of the second needle 30b is in the first needle position, but is configured to allow the second ferrule 92b to operatively couple to the second needle tip 31b of the second needle 30b when the second needle 30b displaces from the second needle position to the first needle position, thereby pulling the second ferrule 92b (and the second portion of suture 95b coupled to the second ferrule 92b) through a second aperture formed in the first portion of tissue or synthetic material disposed within the first tissue bite area 100a. One or more locking or release mechanisms may be disposed at or adjacent to (and/or remote from) the second ferrule recess 91b to selectively retain and release the second ferrule 92b.

With reference to FIG. 3B, a second suture channel 96b may be disposed in a second portion of the distal transverse surface 98 of the receiver portion 74, and the second suture channel 96b may extend from a distal end of the distal portion 94a of the second needle aperture 90b to the distal end of the first suture aperture 99a, and a portion of the second portion of suture 95b may be disposed within the second suture channel 96b such that a second portion of the second portion of suture 95b may extend through the first suture aperture 99a. A third portion of the second portion of suture 95b may extend through the proximal end of the first suture aperture 99a. A fourth portion of the second portion of suture 95b may be disposed along or across a second portion of the plate 110 so that the user may be able to determine the tension of the second portion of suture 95b by a visual inspection of the fourth portion of the second portion of suture 95b. The fourth portion of the second portion of suture 95b may also be disposed offset from the second portion of the plate 110 for the user to determine the tension of the second portion of suture 95b by a visual inspection.

As illustrated in FIG. 3B, the distal end assembly 24 may include a third needle aperture 90c, and the third needle aperture 90c may be identical to, and radially offset from, the second needle aperture 90b (and the first needle aperture 90a). That is, the third needle aperture 90c may be aligned with and configured to receive a portion of the third needle 30c, and the third needle aperture 90c may be aligned with the third needle axis 62c.

A proximal portion 93c of the third needle aperture 90c may extend through a third portion of the base portion 70 and a distal portion 94c of the third needle aperture 90c may extend through a third portion of the receiver portion 74. So configured, when the third needle 30c is in the first needle position of FIG. 1F, the third needle tip 31c at the distal end 66c of the third needle 30c may be disposed within the proximal portion 93c of the third needle aperture 90c and may be disposed proximal to the distal end 78 of the base portion 70. Further, when the third needle 30c is displaced from the first needle position to the second needle position of FIG. 4D, the third needle 30c, and the third needle tip 31c, extends distally along the third needle axis 62c until the third needle tip 31c is disposed within the distal portion 94c of the third needle aperture 90c disposed in the receiver portion 74. When the third needle 30c is in the second needle position, the third needle tip 31c is disposed distal to the proximal end 87 of the receiver portion 74.

When the third needle 30c extends from the first needle position to the second needle position, the third needle tip 31c extends through a second tissue bite area 100b, illustrated in FIGS. 3A and 3E. The second tissue bite area 100b may be partially defined by a third portion of the proximal transverse surface 102 of the receiver portion 74 disposed at the proximal end 87 of the receiver portion 74. The second tissue bite area 100b may be further defined by a third portion of the distal transverse surface 104 of the base portion 70 disposed at the distal end 78 of the base portion 70. The second tissue bite area 100b may be further defined by a first lateral surface 114 of the first column 86a and a second lateral surface 112 of the third column 86c. So configured, when a second portion of tissue or synthetic material may be disposed within the second tissue bite area 100b, the third needle tip 31c of the third needle 30c may extend through the second portion of tissue or synthetic material when the third needle 31c extends from the first needle position to the second needle position.

Also in the second needle position, the third needle tip 31c may be disposed within a third ferrule recess 91c that is formed in all or a portion of the distal portion 94c of the third needle aperture 90c disposed in the receiver portion 74, and the third ferrule recess 91c may be identical or substantially identical to the first ferrule recess 91a. That is, when the third needle tip 31c is disposed within the third ferrule recess 91c, the third needle tip 31c operatively engages (or is configured to engage) a third ferrule 92c that is disposed in the third ferrule recess 91c. The third ferrule 92c may be identical to the first ferrule 92a and may be any ferrule that is configured to be secured to a third portion of suture 95c extending from a distal end of the third ferrule 92c and that may be configured to be selectively coupled to and uncoupled from the third needle tip 31c.

The third ferrule recess 91c may be sized and configured to retain the third ferrule 92c when the third needle tip 31c of the third needle 30c is in the first needle position, but is configured to allow the third ferrule 92c to operatively couple to the third needle tip 31c of the third needle 30c when the third needle 30c displaces from the second needle position to the first needle position, thereby pulling the third ferrule 92c (and the third portion of suture 95c coupled to the third ferrule 92c) through a third aperture formed in the second portion of tissue or synthetic material disposed within the second tissue bite area 100b. One or more locking or release mechanisms may be disposed at or adjacent to (and/or remote from) the third ferrule recess 91c to selectively retain and release the third ferrule 92c.

With reference to FIG. 3B, a third suture channel 96c may be disposed in a third portion of the distal transverse surface 98 of the receiver portion 74, and the third suture channel 96c may extend from a distal end of the distal portion 94c of the third needle aperture 90c to a distal end of a second suture aperture 99b, and a portion of the third portion of suture 95c may be disposed within the third suture channel 96c such that a second portion of the third portion of suture 95c may extend through the second suture aperture 99b. A third portion of the third portion of suture 95c may extend through a proximal end of the second suture aperture 99b that may be disposed at a proximal portion of the housing portion 14. The proximal end of the second suture aperture 99b may be disposed at a corresponding aperture in the plate 110, and a fourth portion of the third portion of suture 95c may be disposed along or across a third portion of the plate 110 so that the user may be able to determine the tension of the third portion of suture 95c by a visual inspection of the fourth portion of the third portion of suture 95c. The fourth portion of the third portion of suture 95c may also be disposed offset from first portion of the plate 110 for the user to determine the tension of the third portion of suture 95c by a visual inspection.

As illustrated in FIG. 3B, the distal end assembly 24 may include a fourth needle aperture 90d, and the fourth needle aperture 90d may be identical to, and radially offset from, the third needle aperture 90c. That is, the fourth needle aperture 90d may be aligned with and configured to receive a portion of the fourth needle 30d, and the fourth needle aperture 90d may be aligned with the fourth needle axis 62d. A proximal portion 93d of the fourth needle aperture 90d may extend through a fourth portion of the base portion 70 and a distal portion 94d of the fourth needle aperture 90d may extend through a fourth portion of the receiver portion 74. So configured, when the fourth needle 30d is in the first needle position of FIG. 1F, the fourth needle tip 31d at the distal end 66b of the fourth needle 30d may be disposed within the proximal portion 93d of the fourth needle aperture 90d and may be disposed proximal to the distal end 78 of the base portion 70. Further, when the fourth needle 30d is displaced from the first needle position to the second needle position of FIG. 4D, the fourth needle 30d, and the fourth needle tip 31d, extends distally along the fourth needle axis 62d until the fourth needle tip 31d is disposed within the distal portion 94d of the fourth needle aperture 90d disposed in the receiver portion 74. When the fourth needle 30d is in the second needle position, the fourth needle tip 31d is disposed distal to the proximal end 87 of the receiver portion 74.

In the second needle position, the fourth needle tip 31d may be disposed within a fourth ferrule recess 91d that is formed in all or a portion of the distal portion 94d of the fourth needle aperture 90d disposed in the receiver portion 74, and when the fourth needle tip 31d is disposed within the fourth ferrule recess 91d, the fourth needle tip 31d operatively engages (or is configured to engage) a fourth ferrule 92d that is disposed in the fourth ferrule recess 91d. The fourth ferrule recess 91d and the fourth ferrule 92d may be identical to the first ferrule recess 91a and the first ferrule 92a, respectively. That is, the fourth ferrule 92d may be any ferrule that is configured to be secured to a fourth portion of suture 95d extending from a distal end of the fourth ferrule 92d and that may be configured to be selectively coupled to and uncoupled from the fourth needle tip 31d.

The fourth ferrule recess 91d may be sized and configured to retain the fourth ferrule 92d when the fourth needle tip 31d of the fourth needle 30d is in the first needle position, but is configured to allow the fourth ferrule 92d to operatively couple to the fourth needle tip 31d of the fourth needle 30d when the fourth needle 30d displaces from the second needle position to the first needle position, thereby pulling the fourth ferrule 92d (and the fourth portion of suture 95d coupled to the fourth ferrule 92d) through a fourth aperture formed in the second portion of tissue or synthetic material disposed within the second tissue bite area 100b. One or more locking or release mechanisms may be disposed at or adjacent to (and/or remote from) the fourth ferrule recess 91d to selectively retain and release the fourth ferrule 92d.

With reference to FIG. 3B, a fourth suture channel 96d may be disposed in a fourth portion of the distal transverse surface 98 of the receiver portion 74, and the fourth suture channel 96d may extend from a distal end of the distal portion 94d of the fourth needle aperture 90d to the distal end of the second suture aperture 99b, and a portion of the fourth portion of suture 95d may be disposed within the fourth suture channel 96d such that a second portion of the fourth portion of suture 95d may extend through the second suture aperture 99b. A third portion of the fourth portion of suture 95d may extend through the proximal end of the second suture aperture 99b. A fourth portion of the fourth portion of suture 95d may be disposed along or across a fourth portion of the plate 110 so that the user may be able to determine the tension of the fourth portion of suture 95d by a visual inspection of the fourth portion of the fourth portion of suture 95d. The fourth portion of the fourth portion of suture 95d may also be disposed offset from the fourth portion of the plate 110 for the user to determine the tension of the fourth portion of suture 95d by a visual inspection.

As illustrated in FIG. 3B, the distal end assembly 24 may include a fifth needle aperture 90e, and the fifth needle aperture 90e may be identical to, and radially offset from, the sixth needle aperture 90f. That is, the fifth needle aperture 90e may be aligned with and configured to receive a portion of the fifth needle 30e, and the fifth needle aperture 90e may be aligned with the fifth needle axis 62e.

A proximal portion 93e of the fifth needle aperture 90e may extend through a fifth portion of the base portion 70 and a distal portion 94e of the fifth needle aperture 90e may extend through a fifth portion of the receiver portion 74. So configured, when the fifth needle 30e is in the first needle position of FIG. 1F, the fifth needle tip 31e at the distal end 66e of the fifth needle 30e may be disposed within the proximal portion 93e of the fifth needle aperture 90e and may be disposed proximal to the distal end 78 of the base portion 70. Further, when the fifth needle 30e is displaced from the first needle position to the second needle position of FIG. 4D, the fifth needle 30e, and the fifth needle tip 31e, extends distally along the fifth needle axis 62e until the fifth needle tip 31e is disposed within the distal portion 94e of the fifth needle aperture 90e disposed in the receiver portion 74. When the fifth needle 30e is in the second needle position, the fifth needle tip 31e is disposed distal to the proximal end 87 of the receiver portion 74.

When the fifth needle 30e extends from the first needle position to the second needle position, the fifth needle tip 31e extends through a third tissue bite area 100c, illustrated in FIGS. 3A and 3E. The third tissue bite area 100c may be partially defined by a fifth portion of the proximal transverse surface 102 of the receiver portion 74 disposed at the proximal end 87 of the receiver portion 74. The third tissue bite area 100c may be further defined by a fifth portion of the distal transverse surface 104 of the base portion 70 disposed at the distal end 78 of the base portion 70. The third tissue bite area 100c may be further defined by a second lateral surface 116 of the first column 86a and a second lateral surface 188 of the second column 86b. So configured, when a third portion of tissue or synthetic material may be disposed within the third tissue bite area 100c, the fifth needle tip 31e of the fifth needle 30e may extend through the third portion of tissue or synthetic material when the third needle 31c extends from the first needle position to the second needle position.

Also in the second needle position, the fifth needle tip 31e may be disposed within a fifth ferrule recess 91e that is formed in all or a portion of the distal portion 94e of the fifth needle aperture 90e disposed in the receiver portion 74, and the fifth ferrule recess 91e may be identical or substantially identical to the first ferrule recess 91a. That is, when the fifth needle tip 31e is disposed within the fifth ferrule recess 91e, the fifth needle tip 31e operatively engages (or is configured to engage) a fifth ferrule 92e that is disposed in the fifth ferrule recess 91e. The fifth ferrule 92e may be identical to the first ferrule 92a and may be any ferrule that is configured to be secured to a fifth portion of suture 95e extending from a distal end of the fifth ferrule 92e and that may be configured to be selectively coupled to and uncoupled from the fifth needle tip 31e.

The fifth ferrule recess 91e may be sized and configured to retain the fifth ferrule 92e when the fifth needle tip 31e of the fifth needle 30e is in the first needle position, but is configured to allow the fifth ferrule 92e to operatively couple to the fifth needle tip 31e of the fifth needle 30e when the fifth needle 30e displaces from the second needle position to the first needle position, thereby pulling the fifth ferrule 92 e (and the fifth portion of suture 95e coupled to the fifth ferrule 92 e) through a fifth aperture formed in the third portion of tissue or synthetic material disposed within the third tissue bite area 100c. One or more locking or release mechanisms may be disposed at or adjacent to (and/or remote from) the fifth ferrule recess 91e to selectively retain and release the fifth ferrule 92e.

With reference to FIG. 3B, a fifth suture channel 96e may be disposed in a fifth portion of the distal transverse surface 98 of the receiver portion 74, and the fifth suture channel 96e may extend from a distal end of the distal portion 94e of the fifth needle aperture 90e to a distal end of a third suture aperture 99c, and a portion of the fifth portion of suture 95e may be disposed within the fifth suture channel 96e such that a second portion of the fifth portion of suture 95e may extend through the third suture aperture 99c. A third portion of the fifth portion of suture 95e may extend through a proximal end of the third suture aperture 99c that may be disposed at a proximal portion of the housing portion 14. The proximal end of the third suture aperture 99c may be disposed at a corresponding aperture in the plate 110, and a fourth portion of the fifth portion of suture 95e may be disposed along or across a third portion of the plate 110 so that the user may be able to determine the tension of the fifth portion of suture 95e by a visual inspection of the fourth portion of the fifth portion of suture 95e. The fourth portion of the fifth portion of suture 95e may also be disposed offset from first portion of the plate 110 for the user to determine the tension of the fifth portion of suture 95e by a visual inspection.

As illustrated in FIG. 3B, the distal end assembly 24 may include a sixth needle aperture 90f, and the sixth needle aperture 90f may be identical to, and radially offset from, the fifth needle aperture 90e. That is, the sixth needle aperture 90f may be aligned with and configured to receive a portion of the sixth needle 30f, and the sixth needle aperture 90f may be aligned with the sixth needle axis 62f. A proximal portion 93f of the sixth needle aperture 90f may extend through a sixth portion of the base portion 70 and a distal portion 94f of the sixth needle aperture 90f may extend through a sixth portion of the receiver portion 74. So configured, when the sixth needle 30f is in the first needle position of FIG. 1F, the sixth needle tip 31f at the distal end 66f of the sixth needle 30f may be disposed within the proximal portion 93f of the sixth needle aperture 90f and may be disposed proximal to the distal end 78 of the base portion 70. Further, when the sixth needle 30f is displaced from the first needle position to the second needle position of FIG. 4D, the sixth needle 30f, and the sixth needle tip 31f, extends distally along the sixth needle axis 62f until the sixth needle tip 31f is disposed within the distal portion 94f of the sixth needle aperture 90f disposed in the receiver portion 74. When the sixth needle 30f is in the second needle position, the sixth needle tip 31f is disposed distal to the proximal end 87 of the receiver portion 74.

In the second needle position, the sixth needle tip 31f may be disposed within a sixth ferrule recess 91f that is formed in all or a portion of the distal portion 94f of the sixth needle aperture 90f disposed in the receiver portion 74, and when the sixth needle tip 31f is disposed within the sixth ferrule recess 91f, the sixth needle tip 31f operatively engages (or is configured to engage) a sixth ferrule 92f that is disposed in the sixth ferrule recess 91f. The sixth ferrule recess 91f and the sixth ferrule 92f may be identical to the first ferrule recess 91a and the first ferrule 92a, respectively. That is, the sixth ferrule 92f may be any ferrule that is configured to be secured to a sixth portion of suture 95f extending from a distal end of the sixth ferrule 92f and that may be configured to be selectively coupled to and uncoupled from the sixth needle tip 31f.

The sixth ferrule recess 91f may be sized and configured to retain the sixth ferrule 92f when the sixth needle tip 31f of the sixth needle 30f is in the first needle position, but is configured to allow the sixth ferrule 92f to operatively couple to the sixth needle tip 31f of the sixth needle 30f when the sixth needle 30f displaces from the second needle position to the first needle position, thereby pulling the sixth ferrule 92f (and the sixth portion of suture 95f coupled to the sixth ferrule 92f) through a sixth aperture formed in the third portion of tissue or synthetic material disposed within the third tissue bite area 100c. One or more locking or release mechanisms may be disposed at or adjacent to (and/or remote from) the sixth ferrule recess 91f to selectively retain and release the sixth ferrule 92f.

With reference to FIG. 3B, a sixth suture channel 96f may be disposed in a sixth portion of the distal transverse surface 98 of the receiver portion 74, and the sixth suture channel 96f may extend from a distal end of the distal portion 94f of the sixth needle aperture 90f to the distal end of the third suture aperture 99c, and a portion of the sixth portion of suture 95f may be disposed within the sixth suture channel 96f such that a second portion of the sixth portion of suture 95f may extend through the third suture aperture 99c. A third portion of the sixth portion of suture 95f may extend through the proximal end of the third suture aperture 99c. A fourth portion of the sixth portion of suture 95f may be disposed along or across a fourth portion of the plate 110 so that the user may be able to determine the tension of the sixth portion of suture 95f by a visual inspection of the fourth portion of the sixth portion of suture 95f. The fourth portion of the sixth portion of suture 95f may also be disposed offset from the fourth portion of the plate 110 for the user to determine the tension of the sixth portion of suture 95f by a visual inspection.

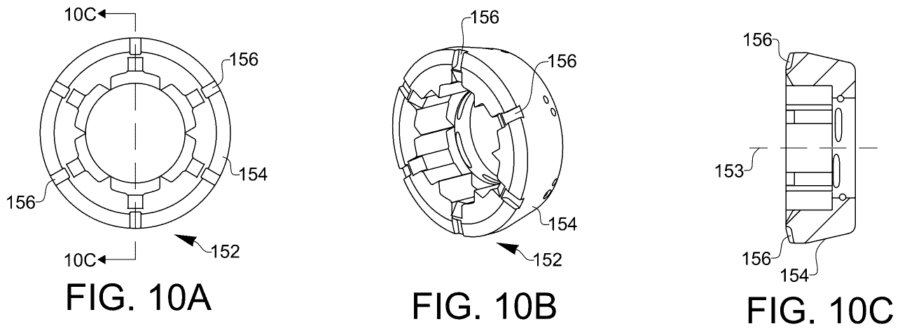

As illustrated in FIGS. 1A and 1B, the suturing device 10 may include a position adjustment assembly 150 that is configured to displace the distal end assembly 24 relative to a portion of the shaft 18, such the distal end 20 of the shaft 18. The position adjustment assembly 150 may include a collar 152 that may be coupled to a portion of the shaft 18, and in some embodiments, the collar 152 may be fixed to the portion of the shaft 18. As illustrated in FIGS. 10A and 10B, the collar 152 may include an annular side wall 154 that may be symmetrically disposed about an axis 153 that may be coaxially aligned with the shaft axis 19. The side wall 154 may include two or more notches 156 that may be disposed along a surface of the side wall 154 that may be at the distal end of the side wall 154. The two or more notches 156 may be disposed symmetrically about the axis 153, and the notches 156 may be disposed at intervals of 180°, 120°, 90°, 60°, or 45°.

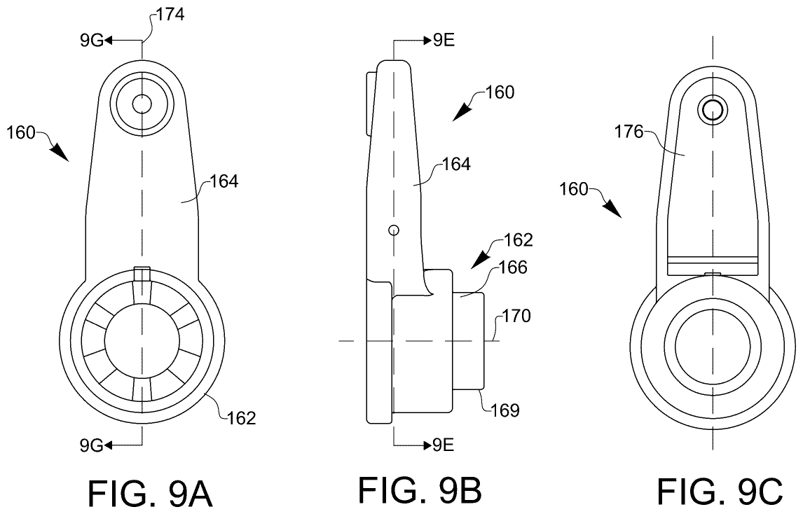

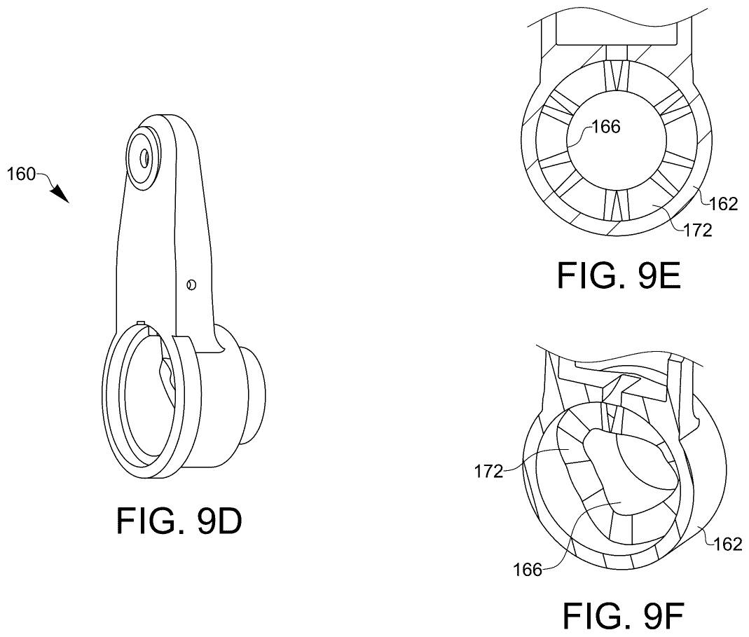

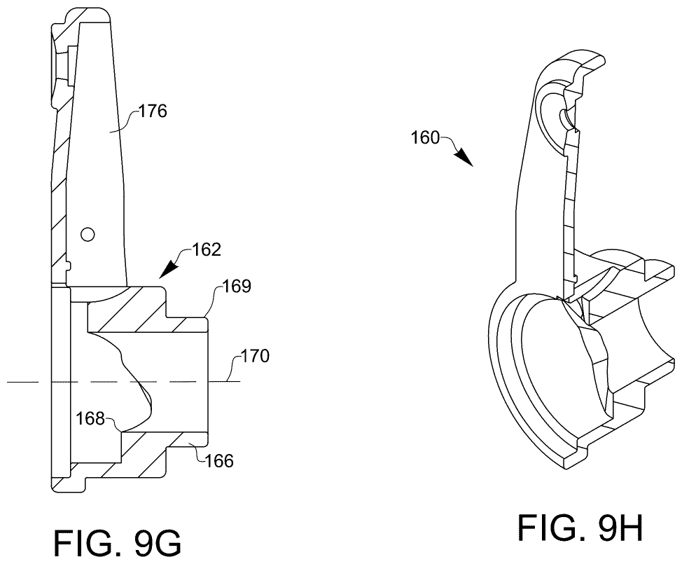

Referring again to FIGS. 1A and 1B, the position adjustment assembly 150 may also include a cam member 160 that may be rotatable relative to the shaft 18 and the collar 152. In particular, the cam member 160 may include a base portion 162 and an extension portion 164. Referring to the cross-sectional view of FIG. 9G, the base portion 162 may include a cylindrical cam portion 166 that extends from a proximal end 168 to a distal end 169 along an axis 170 that may be coaxially aligned with the shaft axis 19. In some embodiments, a portion of the shaft 18 may be received in the aperture formed by the inner surface of the cam portion 162. A cam surface 172 may define the proximal end 168 of the cam portion 166. The cam surface 172 may be irregularly shaped and/or contoured such that the cam surface 172 may vary along the X-axis of the reference coordinate system of FIG. 1F. More specifically, a first longitudinal distance (taken parallel to the axis 170) between a first point on the cam surface 172 and a first point on the distal end 169 of the cam portion 166 (or a first point on a Y-Z plane proximal to the cam surface 172) is different than a second longitudinal distance (taken parallel to the axis 170) between a second point on the cam surface 172 and a second point on the distal end 169 of the cam portion 166 (or a second point on the Y-Z plane proximal to the cam surface 172).

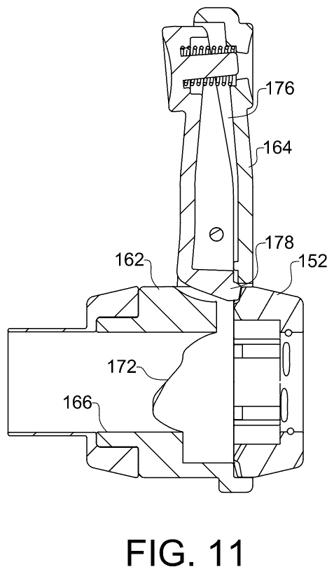

The extension portion 164 may extend from a portion of the base portion 162, and the extension portion may extend from a first end to a second end along an axis 174, with the first end being at or adjacent to the base portion 162. A recess 176 may be disposed on a side of the extension portion 164, and a locking lever 178 may be at least partially disposed within the recess 176. The locking lever 178 may be elongated and may extend from a first end, which may be adjacent to the first end of the extension portion 164, to a second end, which may be adjacent to the second end of the extension portion 164. The locking lever 178 may include a locking tab 180 at the first end. The locking lever 178 may pivot about an axis that in normal to the axis 174 from a first position to a second position, and a sprint may bias the locking lever 178 into the second position. In the first position, the locking tab 180 of the locking lever 178 may be remote from an aperture formed at the first end of the extension portion 164, and in a second position, at least an end portion of the locking tab 180 of the locking lever 178 extends through the aperture formed at the first end of the extension portion 164, as illustrated in FIG. 11.

To rotate the cam member 160 about the collar 152, a user applies pressure to the second end of the locking lever 178 to pivot the locking lever 178 from the second position to the first position, and the cam member 160 is free to rotate relative to the collar 152 (and the shaft 18) about the axis 170. However, due to the bias of the locking lever 178 into the second position, when the locking tab 180 reaches any of the notches 176 disposed on the side wall 154 of the collar 152, the locking tab 180 is received into the notch 176, which prevents further rotation of the cam member 160 is free to rotate relative to the collar 152. The locking lever may then be pivoted into the first position for further rotation relative to the collar 152.

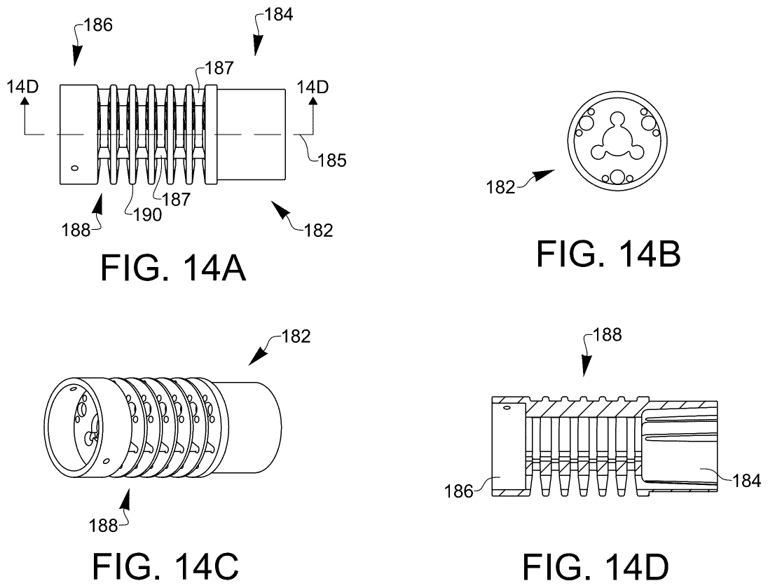

Referring again to FIGS. 1A and 1B, the position adjustment assembly 150 may also include a flexible joint member 182 that may be disposed between the distal end assembly 24 and a portion of the shaft 18, such as the distal end 20 of the shaft 18. With reference to FIGS. 14A to 14C, the flexible joint member 182 may extend from a proximal end to a distal end along an axis 185 that is coaxially aligned with the shaft axis 19. A proximal portion 184 at the proximal end of the flexible joint member 182 may couple the proximal end of the flexible joint member 182 to the distal end 20 of the shaft 18. In addition, a distal portion 186 at the distal end of the flexible joint member 182 may couple the distal end of the flexible joint member 182 to the proximal end 68 of the distal end assembly 24. A hinge portion 188 may be disposed between the proximal end of the distal portion 186 and the distal end of the proximal portion 184. The hinge portion 188 may include two or more longitudinal column members 187 that may extend from the proximal end of the distal portion 186 and the distal end of the proximal portion 184. The hinge portion 188 may also include a plurality of disks 190 that each extend from a portion of the two or more column members 187, and each of the plurality of disks 190 are planar or substantially planar and are aligned and offset along the axis 185.

The hinge portion 188 may be made from a resilient and/or flexible material such that when the proximal portion 184 is fixedly coupled to the distal end 20 of the shaft 18, a force applied to the distal portion 186 (in a direction normal to the shaft axis 19) will displace the distal portion 186 in the direction of the force. For example, the hinge portion 188 may be made from a polymer material. In some embodiments, the proximal portion 184, the distal portion 186, and the hinge portion 188 may be a single, unitary part that may be injection molded.

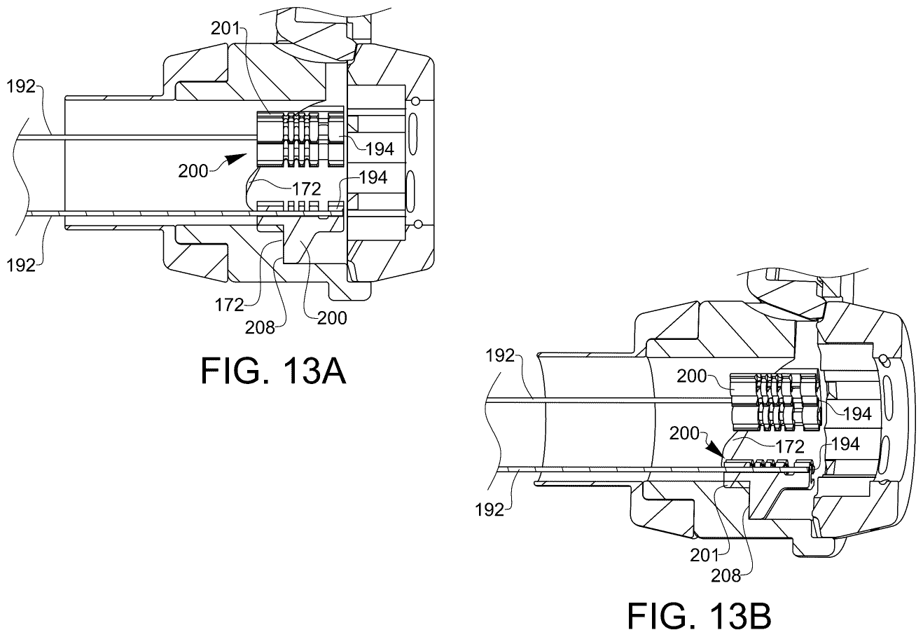

Referring to FIG. 4A, the position adjustment assembly 150 may further include two or more adjustment wires 192 that may each extend from a proximal end 194 (shown in FIG. 13A) to a distal end 196 along an axis that may be parallel to the shaft axis 19. The distal end of 196 of each of the adjustment wires 192 may be coupled to a portion of the proximal end 68 of the distal end assembly 24. In some embodiments, the distal end of 196 of each of the adjustment wires 192 may be spherical and disposed within a recess formed in a portion of the proximal end 68 of the distal end assembly 24. In some embodiments, three adjustment wires 192 are disposed symmetrically about the shaft axis 19 at 120° intervals.

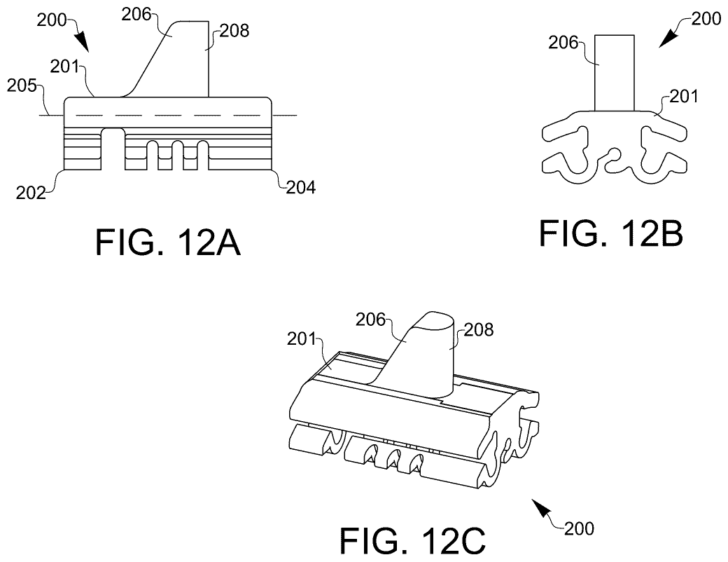

With reference to FIGS. 13A and 13B, which are partial cross-sectional views of a portion of the position adjustment assembly 150, each of the proximal ends 194 of the adjustment wires 192 are coupled to a cam follower member 200. As illustrated in FIGS. 12A to 12C, each of the cam follower members 200 includes a body 201 that extends from a proximal end 202 to distal end 204 along an axis 205 that is parallel to the shaft axis 19. The body 201 may be secured by a portion of the shaft 18 to confine the displacement of the body 201 along the axis 205. For example, the body 201 may include a notch on each lateral side of the body 201 and that may extend parallel to the axis 205, and each notch may receive a corresponding lateral edge (not shown) of an elongated slot formed in the shaft 18, thereby allowing the body 201 to displace along the axis 206 within the slot formed in the shaft 18.

Each of the cam follower members 200 may include a cam projection 206 that extends from a portion of the body 201 in a direction generally normal to the axis 205, and the cam projection 206 may be disposed between the proximal end 202 and the distal end 204. The cam projection 206 may have a contact surface 208 that may extend at (or substantially at) a right angle to the axis 205, and the contact surface 208 may be configured to contact the cam surface 172 (see FIGS. 13A and 13B) of the cam member 160. In some embodiments, the contact surface 208 may be biased into contact with the cam surface 172 such that as the cam member 160 is rotated about the axis 170, the contoured shape of the cam surface 172 displaces each of the cam follower members 200 along the corresponding axes 205 (within the slots formed in the shaft 18, for example). This longitudinal displacement of the cam follower members 200 also displaces the corresponding adjustment wires 192 that have a proximal end 194 secured to the body 201 of each of the cam follower members 200. Thus, as the adjustment wires 192 are displaced along their corresponding axis, the distal end 196 of each of the adjustment wires 192 provides a force on the portion of the proximal end 68 of the distal end assembly 24, thereby causing the bending or flexing of the hinge portion 188 of the flexible joint member 182. The notches 176 of the collar 152 may correspond in position to desired displacements of the distal ends 196 of the adjustment wires 192 such that (a) when the locking tab 180 of the locking lever 178 is received into a first notch 176, the distal end assembly 24 is displaced or pivoted from the shaft axis 19 in a first direction; (b) when the locking tab 180 of the locking lever 178 is received into a second notch 176, the distal end assembly 24 is displaced or pivoted from the shaft axis 19 in a second direction; (c) when the locking tab 180 of the locking lever 178 is received into a third notch 176, the distal end assembly 24 is displaced or pivoted from the shaft axis 19 in a third direction; (d) when the locking tab 180 of the locking lever 178 is received into a fourth notch 176, the distal end assembly 24 is displaced or pivoted from the shaft axis 19 in a fourth direction; (e) when the locking tab 180 of the locking lever 178 is received into a fifth notch 176, the distal end assembly 24 is displaced or pivoted from the shaft axis 19 in a fifth direction; and (a) when the locking tab 180 of the locking lever 178 is received into a sixth notch 176, the distal end assembly 24 is displaced or pivoted from the shaft axis 19 in a sixth direction. Thus, a user may rotate the cam member 160 to provide fine-tuned pre-set displacement of the distal end assembly 24 relative to the distal end 20 of the shaft 18 to allow the user to more easily position the distal end assembly 24 at the treatment area.

Referring to FIG. 1F, the suturing device 10 may include the instrument conduit 80 that may extend through one or more portions of the suturing device 10, and the instrument conduit 80 may be configured to receive an instrument, such as an endoscope or an ICE probe. For example, a portion of the instrument conduit 80 may be disposed through portions of the distal end assembly 24, as previously described. In addition, a portion of the instrument conduit 80 may be disposed through all or a portion of the interior portion 109 of the shaft 18. In some embodiments, a shaft insert 122 may extend through the interior portion 109 of the shaft 18, and the shaft insert 122 may have an instrument aperture 124 that may define the portion of the instrument conduit 80. As illustrated in the cross-sectional view of FIG. 1G, the shaft insert 122 may also have a plurality of lateral apertures 125 that may each be configured to receive a corresponding one of the first needle 30a, the second needle 30b, the third needle 30c, the fourth needle 30d, the fifth needle 30e, and the sixth needle 30f. Turning again to FIG. 1F, a distal end of the access tube 17 may be coupled to a proximal end of the shaft insert 122, and the distal end of the access tube 17 may be inserted into a proximal end of the instrument aperture 124 of the shaft insert 122. The access tube 17 may extend through the interior of the housing portion 14, and all or a portion of an interior of the access tube 17 may define a further portion of the instrument conduit 80. A proximal end of the access tube 17 may be coupled to a portion of the plate 110 that is adjacent to a plate insert aperture 126 such that plate insert aperture 126 opens to a proximal end of the instrument conduit 80. While the instrument conduit 80 may have one or more diameters, each of the one or more diameters may be configured to receive the instrument, such as the ICE probe to allow the distal end of the ICE probe to be inserted into the proximal end of the instrument conduit 80, extend through the length of the instrument conduit 80, and exit through the distal end of the instrument conduit 80, and vice versa. In use, the distal end of the ICE probe may be inserted through the plate insert aperture 126 and advanced through the instrument conduit 80 until the distal end of the ICE probe exits the distal end of the instrument conduit 80 at the distal end 69 of the distal end assembly 24 and extends to a desired treatment area.

Once the distal end of the ICE probe 134 is disposed at the desired treatment area, the user may displace the blocking lever 12 from the disengaged position (illustrated in FIG. 2B) to the engaged position (illustrated in FIG. 2A), and the engagement portion 15 of the blocking lever 12 contacts the portion of the access tube 17 (illustrated in FIG. 2A) to frictionally engage a portion of the ICE probe 134 (see FIG. 2C) to prevent further movement of the ICE probe 134 relative to the instrument conduit 80. In some embodiments, the engagement portion 15 of the blocking lever 12 extends through a portion of the access tube 17, such as an access aperture in the access tube 17 (illustrated in FIG. 2A), such that the engagement portion 15 directly engages the portion on an outer surface of the ICE probe 134. Thus, the distal end of the ICE probe 134 may be maintained in a desired position to view a specific area during the procedure. When it is desired to displace the distal end of the ICE probe 134, such as to fine tune the position of the distal end of the ICE probe 134, the user may release the blocking lever 12 to displace the blocking lever 12 from the engaged position (illustrated in FIG. 2A) to the disengaged (illustrated in FIG. 2B) such that the engagement portion 15 of the blocking lever 12 no longer contacts the portion of the access tube 17 or the portion of the ICE probe 134.

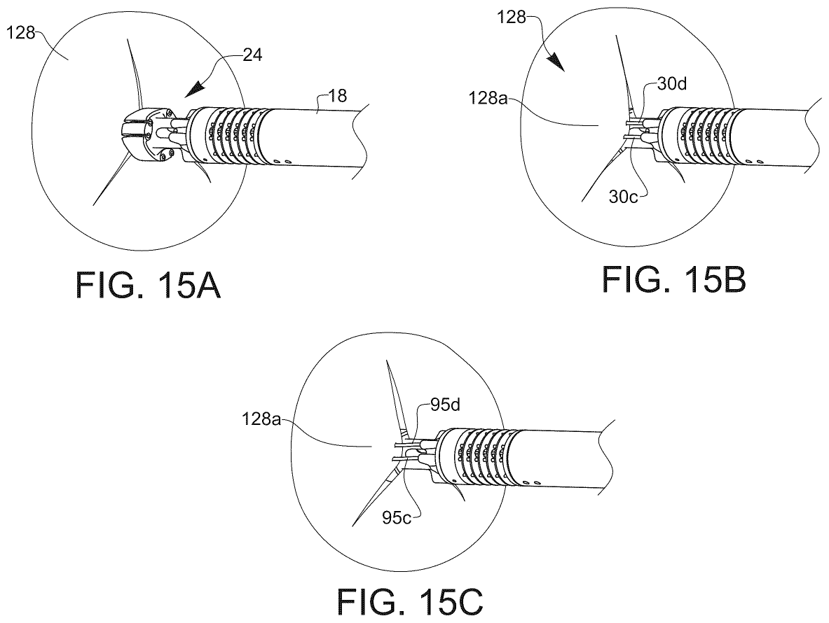

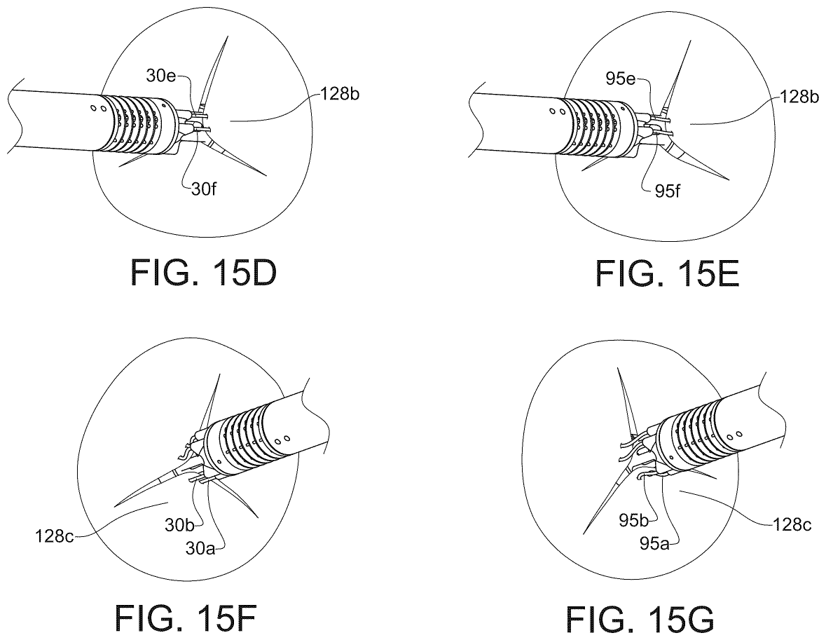

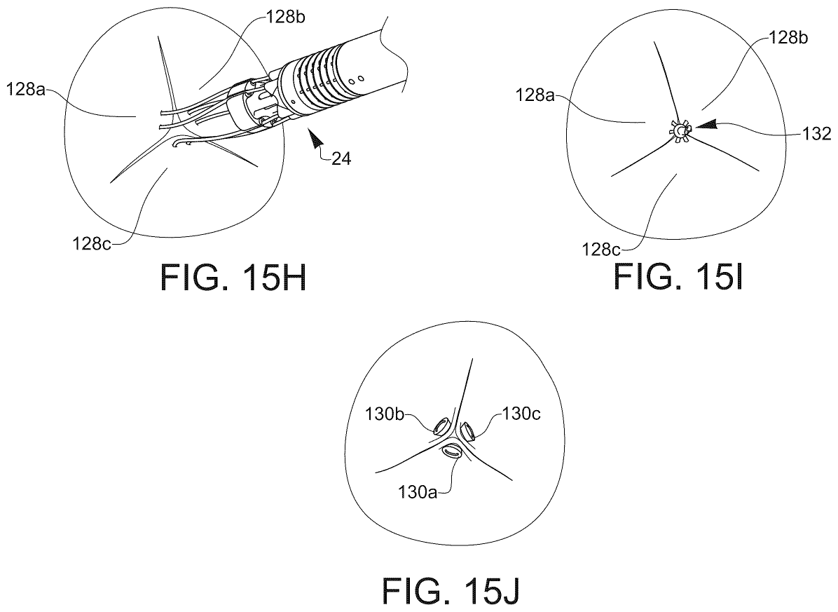

The suturing device 10 may be used in any suitable medical procedure that requires suturing. For example, the device may be used in the repair of a tricuspid valve 128, as illustrated in FIGS. 15A to 15J. In this procedure, after positioning the distal end assembly 24 at or adjacent to the treatment area, and after positioning the distal end of the ICE probe 134 to view the treatment area as described above, a user may position the distal end assembly 24 of the suturing device 10 adjacent to a first valve leaflet 128a, as illustrated in FIG. 15A, and may position a portion of the first valve leaflet 128a in the second tissue bite area 100b. In some embodiments, the user may use the position adjustment assembly 150 to more precisely position the distal end assembly 24 without the need to significantly displace the distal end 20 of the shaft 18, which may be difficult in the space available in a minimally-invasive surgical procedure. The user may then displace the third actuation lever 36c and the fourth actuation lever 36d from the first lever position (illustrated in FIG. 5A) to the second lever position (illustrated in FIG. 5B) such that the third needle 30c and the fourth needle 30d extend through corresponding portions of the first valve leaflet 128a, as illustrated in FIG. 15B. When the third needle 30c and the fourth needle 30d reach the second needle position (of FIG. 4D), the third needle tip 31c and the fourth needle tip 31d operatively engage the third ferrule 92c and the fourth ferrule 92d, respectively. When the third actuation lever 36c and the fourth actuation lever 36d are displaced from the second lever position (illustrated in FIG. 5B) to the first lever position (illustrated in FIG. 5A), the retracting third needle tip 31c and the fourth needle tip 31d retain the third ferrule 92c and the fourth ferrule 92d, respectively, and the third portion of suture 95d and the fourth portion of suture 95d are also retraced through the corresponding portions of the first valve leaflet 128a, as illustrated in FIG. 15C. In some embodiments, an end portion of the third portion of suture 95d is integrally formed with an end portion of the fourth portion of suture 95d such that a half-mattress suture is formed, and a first pledget 130a (see FIG. 15J) is disposed between the tissue of the first valve leaflet 128a and the end portions of the third and fourth portions of suture 95c. Prior to application, the first pledget 130a may be disposed in a recess formed in the distal transverse surface 98, as illustrated in FIG. 4B. In some embodiments, the third actuation lever 36c and the fourth actuation lever 36d may be displaced from the first lever position to the second lever position simultaneously or at different times, and the third actuation lever 36c and the fourth actuation lever 36d may be displaced from the second lever position to first lever position simultaneously or at different times.

The user may next position the distal end assembly 24 of the suturing device 10 adjacent to a second valve leaflet 128b, and may position a portion of the second valve leaflet 128b in the third tissue bite area 100c. The user may then displace the fifth actuation lever 36e and the sixth actuation lever 36f from the first lever position (illustrated in FIG. 5A) to the second lever position (illustrated in FIG. 5B) such that the fifth needle 30e and the sixth needle 30f extend through corresponding portions of the second valve leaflet 128b, as illustrated in FIG. 15D. When the fifth needle 30e and the sixth needle 30f reach the second needle position (of FIG. 4D), the fifth needle tip 31e and the sixth needle tip 31f operatively engage the fifth ferrule 92e and the sixth ferrule 92f, respectively. When the fifth actuation lever 36e and the sixth actuation lever 36f are displaced from the second lever position (illustrated in FIG. 5B) to the first lever position (illustrated in FIG. 5A), the retracting fifth needle tip 31e and the sixth needle tip 31f retain the fifth ferrule 92e and the sixth ferrule 92f, respectively, and the fifth portion of suture 95e and the sixth portion of suture 95f are also retraced through the corresponding portions of the second valve leaflet 128b, as illustrated in FIG. 15E. In some embodiments, an end portion of the fifth portion of suture 95e is integrally formed with an end portion of the sixth portion of suture 95f such that a half-mattress suture is formed, and a second pledget 130b (see FIG. 15J) is disposed between the tissue of the second valve leaflet 128b and the end portions of the fifth and sixth portions of suture 95e, 95f. Prior to application, the second pledget 130b may be disposed in a recess formed in the distal transverse surface 98, as illustrated in FIG. 4B. In some embodiments, the fifth actuation lever 36e and the fourth actuation lever 36d may be displaced from the first lever position to the second lever position simultaneously or at different times, and the fifth actuation lever 36e and the fourth actuation lever 36d may be displaced from the second lever position to first lever position simultaneously or at different times.

The user may then position the distal end assembly 24 of the suturing device 10 adjacent to a third valve leaflet 128c, and may position a portion of the third valve leaflet 128c in the first tissue bite area 100a. The user may then displace the first actuation lever 36a and the second actuation lever 36f from the first lever position (illustrated in FIG. 5A) to the second lever position (illustrated in FIG. 5B) such that the first needle 30a and the second needle 30b extend through corresponding portions of the third valve leaflet 128c, as illustrated in FIG. 15F. When the first needle 30a and the second needle 30b reach the second needle position (of FIG. 4D), the first needle tip 31a and the second needle tip 31b operatively engage the first ferrule 92a and the second ferrule 92b, respectively. When the first actuation lever 36e and the second actuation lever 36f are displaced from the second lever position (illustrated in FIG. 5B) to the first lever position (illustrated in FIG. 5A), the retracting first needle tip 31a and the second needle tip 31b retain the first ferrule 92a and the second ferrule 92b, respectively, and the first portion of suture 95a and the second portion of suture 95b are also retraced through the corresponding portions of the third valve leaflet 128c, as illustrated in FIG. 15G. In some embodiments, an end portion of the first portion of suture 95a is integrally formed with an end portion of the second portion of suture 95b such that a half-mattress suture is formed, and a third pledget 130c (see FIG. 15J) is disposed between the tissue of the third valve leaflet 128c and the end portions of the first and second portions of suture 95a, 95b. Prior to application, the third pledget 130c may be disposed in a recess formed in the distal transverse surface 98, as illustrated in FIG. 4B. In some embodiments, the first actuation lever 36e and the fourth actuation lever 36d may be displaced from the first lever position to the second lever position simultaneously or at different times, and the first actuation lever 36e and the fourth actuation lever 36d may be displaced from the second lever position to first lever position simultaneously or at different times.

With the first, second, third, fourth, fifth, and sixth portions of suture 95a, 95b, 95c, 95d, 95e, 95f placed as described, the distal end assembly 24 of the suturing device 10 may be retracted away from the tricuspid valve 128, as illustrated in FIG. 15H. The first, second, third, fourth, fifth, and sixth portions of suture 95a, 95b, 95c, 95d, 95e, 95f may then be secured, such as by a mechanical fastener 132, as illustrated in FIG. 15I. While in this embodiment, the first valve leaflet 128a is disposed in the second tissue bite area 100b, the second valve leaflet 128b is disposed in the third tissue bite area 100c, and the first valve leaflet 128b is disposed in the first tissue bite area 100a, any suitable valve leaflet 128 may be disposed in any suitable bite area 100 in any order during the procedure. Thus, device provides a non-running, straight needle device intended to provide the sutures for an “edge-edge type” structural repair in which the needles can be actuated individually or separately to pierce the tricuspid valve leaflets and place three independent half-mattress stitches. This device allows for a more streamlined application of sutures to the fragile tissue of the tricuspid valve leaflets in a minimally-invasive procedure in which visibility of the treatment area and maneuverability of the device are each severely restricted.

Various advantages of the suturing device have been discussed above. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A suturing device comprising:
a housing having one or more surfaces that define an interior portion of the housing;
an elongated shaft extending from a proximal end to a distal end about a shaft axis, the shaft being coupled to a first portion of the housing, the shaft having an internal surface that extends from the proximal end to the distal end and that defines an internal portion of the shaft;
a distal end assembly extending along the shaft axis from a proximal end to a distal end, wherein a portion of the proximal end of the distal end assembly is coupled to the distal end of the shaft, the distal end assembly including a receiver portion disposed at a distal end of the distal end assembly, the receiver portion including a distal end of a first needle aperture, a distal end of a second needle aperture, a distal end of a third needle aperture, and a distal end of a fourth needle aperture;
a first needle extending from a proximal end to a distal end about a first needle axis, the first needle having a first needle tip at the distal end of the first needle, wherein at least a portion of the first needle is disposed within the internal portion of the shaft, the first needle being displaceable about the first needle axis between a first needle position, in which the first needle tip is disposed offset from the distal end of the first needle aperture, and a second needle position, in which the first needle tip is disposed within a portion of the distal end of the first needle aperture;
a second needle extending from a proximal end to a distal end about a second needle axis, the second needle having a second needle tip at the distal end of the second needle, wherein at least a portion of the second needle is disposed within the internal portion of the shaft, the second needle being displaceable about the second needle axis between a first needle position, in which the second needle tip is disposed offset from the distal end of the second needle aperture, and a second needle position, in which the second needle tip is disposed within a portion of the distal end of the second needle aperture;
a third needle extending from a proximal end to a distal end about a third needle axis, the third needle having a third needle tip at the distal end of the third needle, wherein at least a portion of the third needle is disposed within the internal portion of the shaft, the third needle being displaceable about the third needle axis between a first needle position, in which the third needle tip is disposed offset from the distal end of the third needle aperture, and a second needle position, in which the third needle tip is disposed within a portion of the distal end of the third needle aperture;
a fourth needle extending from a proximal end to a distal end about a fourth needle axis, the fourth needle having a fourth needle tip at the distal end of the fourth needle, wherein at least a portion of the fourth needle is disposed within the internal portion of the shaft, the fourth needle being displaceable about the fourth needle axis between a first needle position, in which the fourth needle tip is disposed offset from the distal end of the fourth needle aperture, and a second needle position, in which the fourth needle tip is disposed within a portion of the distal end of the fourth needle aperture; and
an actuation assembly comprising:
a first actuation lever that includes a first base portion and a first lever portion, wherein the first lever portion is elongated and extends from a first end to a second end, the first end being coupled to a first portion of the first base portion, the first base portion being pivotable about a first pivot axis that extends through a second portion of the first base portion such that the first actuation lever pivots about the first pivot axis relative to the housing, wherein at least a portion of the first base portion is disposed in the interior portion of the housing and at least a portion of the first lever portion is disposed exterior to the interior portion of the housing, and
wherein the proximal end of the first needle is coupled to a third portion of the first base portion, and the first lever portion is configured to be displaced by a user to pivot the first actuator lever between a first lever position and a second lever position, wherein in the first lever position, the first needle is in the first needle position, and in the second lever position, the first needle is in the second needle position;
a second actuation lever that includes a second base portion and a second lever portion, wherein the second lever portion is elongated and extends from a first end to a second end, the first end being coupled to a first portion of the second base portion, the second base portion being pivotable about a second pivot axis that extends through a second portion of the second base portion such that the second actuation lever pivots about the second pivot axis relative to the housing, wherein at least a portion of the second base portion is disposed in the interior portion of the housing and at least a portion of the second lever portion is disposed exterior to the interior portion of the housing, and
wherein the proximal end of the second needle is coupled to a third portion of the second base portion, and the second lever portion is configured to be displaced by a user to pivot the second actuator lever between a first lever position and a second lever position, wherein in the first lever position, the second needle is in the first needle position, and in the second lever position, the second needle is in the second needle position;
a third actuation lever that includes a third base portion and a third lever portion, wherein the third lever portion is elongated and extends from a first end to a second end, the first end being coupled to a first portion of the third base portion, the third base portion being pivotable about a third pivot axis that extends through a second portion of the third base portion such that the third actuation lever pivots about the third pivot axis relative to the housing, wherein at least a portion of the third base portion is disposed in the interior portion of the housing and at least a portion of the third lever portion is disposed exterior to the interior portion of the housing, and
wherein the proximal end of the third needle is coupled to a third portion of the third base portion, and the third lever portion is configured to be displaced by a user to pivot the third actuator lever between a first lever position and a second lever position, wherein in the first lever position, the third needle is in the first needle position, and in the second lever position, the third needle is in the second needle position;
a fourth actuation lever that includes a fourth base portion and a fourth lever portion, wherein the fourth lever portion is elongated and extends from a first end to a second end, the first end being coupled to a first portion of the fourth base portion, the fourth base portion being pivotable about a fourth pivot axis that extends through a second portion of the fourth base portion such that the fourth actuation lever pivots about the fourth pivot axis relative to the housing, wherein at least a portion of the fourth base portion is disposed in the interior portion of the housing and at least a portion of the fourth lever portion is disposed exterior to the interior portion of the housing, and
wherein the proximal end of the fourth needle is coupled to a third portion of the fourth base portion, and the fourth lever portion is configured to be displaced by a user to pivot the fourth actuator lever between a first lever position and a second lever position, wherein in the first lever position, the fourth needle is in the first needle position, and in the second lever position, the fourth needle is in the second needle position.

2. The suturing device of claim 1, further comprising:
an access tube having a first portion that extends from a proximal end at a second portion of the housing to a distal end, the access tube being configured to receive a portion of an elongated scope;
a blocking lever pivotably coupled to a third portion of the housing, the blocking lever having an elongated engagement arm having an engagement portion disposed at or adjacent to an end of the engagement arm,
wherein the blocking lever pivots from a first blocking lever position, in which the engagement portion is disposed remote from a portion of the first portion of the access tube, to a second blocking lever position, in which a portion of the engagement portion is disposed:
(a) in contact with the portion of the first portion of the access tube such that the portion of the first portion of the access tube is configured to contact and frictionally engage a portion of an external surface of the scope to prevent longitudinal displacement of the scope relative to the access tube; or
(b) within an engagement aperture formed in the portion of the first portion of the access tube such that the portion of the engagement portion is configured to contact and frictionally engage a portion of an external surface of the scope to prevent longitudinal displacement of the scope relative to the access tube.

3. The suturing device of claim 2, wherein the blocking lever includes a lever portion that outwardly extends from an aperture formed in a grip portion of the housing, and the lever portion is configured to be pivoted from the first blocking lever position to the second blocking lever position by contact of a finger of a hand of a user that is grasping the grip portion of the housing.

4. The suturing device of claim 2, wherein the distal end of the first portion of access tube is disposed within a portion of the interior portion of the shaft.

5. The suturing device of claim 1, wherein the shaft is coupled to the first portion of the housing at or adjacent to the proximal end of the shaft such that the proximal end of the shaft is disposed in the interior portion of the housing.

6. The suturing device of claim 1, wherein each of the first pivot axis, the second pivot axis, the third pivot axis, and the fourth pivot axis is normal to the shaft axis.

7. The suturing device of claim 1, wherein the first base portion is planar and normal to the first pivot axis, and the second base portion is planar and normal to the second pivot axis, and the first base portion is offset from the second base portion along the first pivot axis.

8. The suturing device of claim 7, wherein the first pivot axis is coaxially aligned with the second pivot axis.

9. The suturing device of claim 8, wherein the third base portion is planar and normal to the third pivot axis, and the fourth base portion is planar and normal to the fourth pivot axis, and the third base portion is offset from the second base portion along the second pivot axis and is offset from the fourth base portion along the fourth pivot axis.

10. The suturing device of claim 9, wherein the third pivot axis is coaxially aligned with the second pivot axis and the fourth pivot axis is coaxially aligned with the third pivot axis.

11. The suturing device of claim 1, wherein each of the first needle axis, the second needle axis, the third needle axis, and the fourth needle axis is parallel to the shaft axis.

12. The suturing device of claim 1, wherein each of the first base portion, the second base portion, the third base portion, and the fourth base portion is disposed within the interior portion of the housing.

13. The suturing device of claim 1, wherein each of the first actuator lever, the second actuator lever, the third actuator lever, and the fourth actuator lever is configured to be pivoted between the first lever position and the second lever position by a user applying a force to a portion of the first lever portion, the second lever portion, the third lever portion, and the fourth lever portion, respectively, that is disposed exterior to the interior portion of the housing.

14. The suturing device of claim 1, wherein each of the first needle axis, the second needle axis, the third needle axis, and the fourth needle axis is parallel to the shaft axis.
